(19) United States
(12) Patent Application Publication
(10) Pub. No.: US 2023/0295086 A1
(43) Pub. Date: Sep. 21, 2023

(54) NOVEL PSILOCIN DERIVATIVES HAVING PRODRUG PROPERTIES

(71) Applicant: COMPASS PATHFINDER LIMITED, Altrincham (GB)

(72) Inventor: Matthias GRILL, Basel (CH)

(21) Appl. No.: 18/021,243

(22) PCT Filed: Aug. 23, 2021
(86) PCT No.: PCT/EP2021/073303

§ 371 Date: Feb. 14, 2023

Related U.S. Application Data
(60) Provisional application No. 63/118,842, filed on Nov. 27, 2020.

(30) Foreign Application Priority Data
Aug. 21, 2020 (DE) 10 2020 121 965.2

(51) Int. Cl. C07D 209/16; C07D 413/12; A61P 25/28

(52) U.S. Cl. CPC C07D 209/16; C07D 413/12; A61P 25/28

(57) ABSTRACT

The present invention provides a novel group of active compounds based on the psychoactive compound psilocin. The psilocin derivatives provided herein exhibit improved pharmacokinetic properties during uptake as compared to psilocin, as well as reduced side effects resulting from the metabolites thus formed. Due to the affinity of the novel psilocin derivatives for the 5-HT_{2A}-receptor, these derivatives are particularly advantageous for use in therapy, e.g., in the treatment of depression or drug addiction.

The present application claims the priority of German patent application DE 10 2020 121 965.2 filed on Aug. 21, 2020 and U.S. provisional application U.S. 63/118,842 filed on Nov. 27, 2020, the contents of which are incorporated herein by reference in their entirety.

### BACKGROUND

In nature, psilocin is found only in the form of its precursor as a phosphate ester called “psilocybin”. In the precursor, the psychoactive compound psilocin, which is sensitive to oxidation, is protected by a phosphate group. Upon uptake in the body, this protective group is cleaved hydrolytically, and the active compound psilocin is released.

To date, all medical and clinical studies use the native psilocybin. It is important to note that the industrial synthesis of psilocybin is a complicated and costly process which, due to the use of carcinogenic catalysts, is to be reviewed critically, at least for GMP applications.

Research on hallucinogens has been experiencing a revival since about 1990.

In 2018, the US health authority FDA approved a study of the company “Compass Pathways”, in which patients with treatment-resistant depression are to be treated with the active compound psilocybin.

Recent studies show promising results for the therapy of cancer-related anxiety conditions and for the withdrawal of nicotine or alcohol.

In particular, the application by so-called “microdosing”, i.e. the administration of small doses, has shifted into the focus of research in recent years. The purpose of this administration form is to avoid eliciting hallucinations and to avoid side effects by using small dosages and long dosage intervals in the range of days or even weeks.

Novel psilocin derivatives, in particular those showing a modified (accelerated or retarded) activity in the human body due to their structure, are of increasing pharmaceutical interest.

As only a limited range of psilocin derivatives has been described in the literature (see, e.g., U.S. Pat. No. 3,075,992 and CH 386,442), none of which has given rise to the development of a successful therapeutic product, there is still an urgent and unmet need for novel psilocin derivatives having improved therapeutic properties.

The present invention addresses this need and provides novel and easily producible psilocin derivatives based on a carbonate or amino acid derivatization. The novel psilocin derivatives provided herein exhibit improved properties which render them highly advantageous for therapeutic use.

### BRIEF DESCRIPTION OF THE DRAWINGS

FIG. 1: Thin layer chromatograms of the starting material psilocin (E), the end product psilocin-4-yl ethyl carbonate (CO3) and the intermediate product psilocin-4-yl-Fmoc-tryptophanate (AS) in chloroform/ethanol 10:1 (left panel) and dichloromethane/methanol 7:3 (right panel), respectively.

FIG. 2: Thin layer chromatograms of the starting material psilocin (E), the end product psilocin-4-yl ethyl carbonate (CO3) and the intermediate product psilocin-4-yl-Fmoc-tryptophanate (AS) in tert-butyl methyl ether/ethanol 8:2 (left panel), hexane/ethyl acetate 7:3 (central panel), and tert-butyl methyl ether/isopropanol 8:2 (right panel), respectively.

FIG. 3: HPLC-MS spectrum of psilocin-4-yl ethyl carbonate from the reaction solution.

FIG. 4: HPLC-MS spectrum of psilocin-4-yl-Fmoc-tryptophanate from the reaction solution.

FIG. 5: Stability of novel psilocin carbonates in HCl. (A) % of parent compound remaining following incubation in 1% HCl solution over 24 hours. (B) % of psilocin liberated from test compounds during incubation in 1% HCl. (C) % of psilocin tert-butylcarbonate remaining following incubation in 1% HCl solution for over 24 hours. (D) % of psilocin liberated from test compounds during incubation in 1% HCl. See Example 9.

FIG. 6: Pharmacokinetics of novel psilocin carbonates in the mouse. (A) Plasma psilocin concentrations following intravenous dosing of mice with test compounds. Data shown as mean±SEM. (B) Plasma psilocin concentrations following oral dosing of mice with test compounds. Data shown as mean±SEM. (C) Plasma psilocin concentrations following intravenous dosing of mice with psilocybin or psilocin-4-yl-ethylcarbonate. Data shown as mean±SEM. (D) Plasma psilocin concentrations following oral dosing of mice with psilocybin or psilocin-4-yl-ethylcarbonate. Data shown as mean±SEM. (E) Plasma psilocin concentrations following intravenous dosing of mice with psilocybin or psilocin-4-yl-tert-butylcarbonate. Data shown as mean±SEM. (F) Plasma psilocin concentrations following oral dosing of mice with psilocybin or psilocin-4-yl-tert-butylcarbonate. Data shown as mean±SEM. (G) Plasma psilocin concentrations following intravenous dosing of mice with psilocybin or psilocin-4-yl-benzylcarbonate. Data shown as mean±SEM. (H) Plasma psilocin concentrations following oral dosing of mice with psilocybin or psilocin-4-yl-benzylcarbonate. Data shown as mean±SEM. See Example 12.

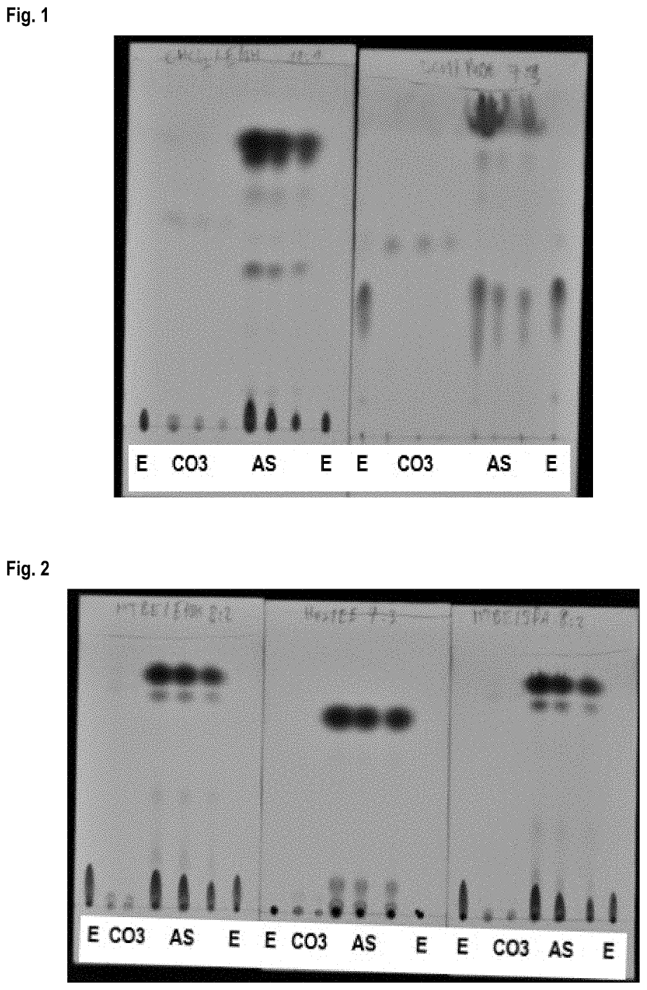

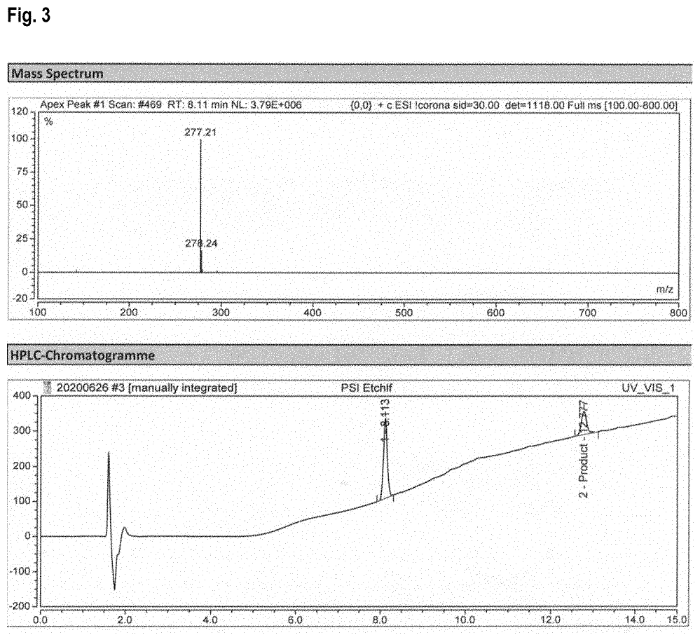

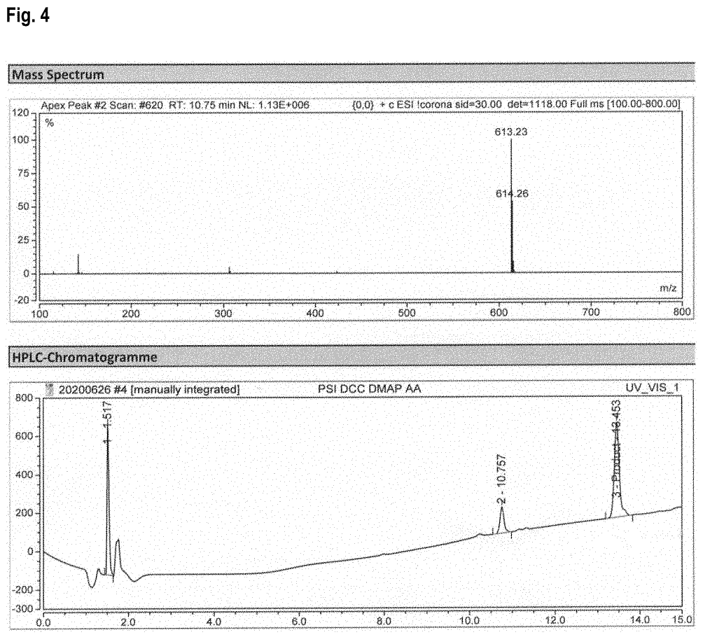

### DETAILED DESCRIPTION

### Definitions

For convenience, certain terms employed in the specification, examples and claims are collected here. Unless defined otherwise, all technical and scientific terms used in this disclosure have the same meanings as commonly understood by one of ordinary skill in the art to which this disclosure belongs.

As used herein, unless explicitly indicated otherwise or contradicted by context, the terms “a”, “an” and “the” are used interchangeably with “one or more” and “at least one”. Thus, for example, a composition comprising “a” compound of formula (I) can be interpreted as referring to a composition comprising “one or more” compounds of formula (I).

The terms “administer,” “administering” or “administration” as used herein refer to administering a compound or pharmaceutically acceptable salt of the compound or a composition or formulation comprising the compound or pharmaceutically acceptable salt of the compound to a patient.

As used herein, the term “alkyl” refers to a monovalent saturated acyclic (i.e., non-cyclic) hydrocarbon group (i.e., a group consisting of carbon atoms and hydrogen atoms) which may be linear or branched. Accordingly, an “alkyl” group does not comprise any carbon-to-carbon double bond or any carbon-to-carbon triple bond. A “C₁₋₁₂ alkyl” denotes an alkyl group having 1 to 12 carbon atoms. Preferred exemplary alkyl groups are methyl, ethyl, propyl (e.g., n-propyl or isopropyl), or butyl (e.g., n-butyl, isobutyl, sec-butyl, or tert-butyl). Unless stated otherwise specifically in the specification, an alkyl group can be optionally substituted.

As used herein, the term “alkylene” refers to an alkanediyl group, i.e. a divalent saturated acyclic hydrocarbon group which may be linear or branched. A “C₁₋₁₂ alkylene” denotes an alkylene group having 1 to 12 carbon atoms. Preferred exemplary alkylene groups are methylene (—CH₂—), ethylene (e.g., —CH₂—CH₂— or —CH(—CH₃)—), propylene (e.g., —CH₂—CH₂—CH₂—, —CH(—CH₂—CH₃)—, —CH₂—CH(—CH₃)—, or —CH(—CH₃)—CH₂—), or butylene (e.g., —CH₂—CH₂—CH₂—CH₂—). Preferred exemplary alkylene groups include methylene, ethylene, propylene, or butylene. Unless stated otherwise specifically in the specification, an alkylene chain can be optionally substituted.

“Alkenyl” or “alkenyl group” refers to a straight or branched hydrocarbon chain having from two to twelve carbon atoms and having one or more carbon-carbon double bonds. Each alkenyl group is attached to the rest of the molecule by a single bond. Alkenyl group comprising any number of carbon atoms from 2 to 12 are included. An alkenyl group comprising up to 12 carbon atoms is a C₂-C₁₂ alkenyl, an alkenyl comprising up to 10 carbon atoms is a C₂-C₁₀ alkenyl, an alkenyl group comprising up to 6 carbon atoms is a C₂-C₆ alkenyl and an alkenyl comprising up to 5 carbon atoms is a C₂-C₅ alkenyl. A C₂-C₅ alkenyl includes C₅ alkenyls, C₄ alkenyls, C₃ alkenyls, and C₂ alkenyls. A C₂-C₆ alkenyl includes all moieties described above for C₂-C₅ alkenyls but also includes C₆ alkenyls. A C₂-C₁₀ alkenyl includes all moieties described above for C₂-C₅ alkenyls and C₂-C₆ alkenyls, but also includes C₇, C₈, C₉ and C₁₀ alkenyls. Similarly, a C₂-C₁₂ alkenyl includes all the foregoing moieties, but also includes C₁₁ and C₁₂ alkenyls. Non-limiting examples of C₂-C₁₂ alkenyl include ethenyl (vinyl), 1-propenyl, 2-propenyl (allyl), iso-propenyl, 2-methyl-1-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 6-heptenyl, 1-octenyl, 2-octenyl, 3-octenyl, 4-octenyl, 5-octenyl, 6-octenyl, 7-octenyl, 1-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, 9-decenyl, 1-undecenyl, 2-undecenyl, 3-undecenyl, 4-undecenyl, 5-undecenyl, 6-undecenyl, 7-undecenyl, 8-undecenyl, 9-undecenyl, 10-undecenyl, 1-dodecenyl, 2-dodecenyl, 3-dodecenyl, 4-dodecenyl, 5-dodecenyl, 6-dodecenyl, 7-dodecenyl, 8-dodecenyl, 9-dodecenyl, 10-dodecenyl, and 11-dodecenyl. Unless stated otherwise specifically in the specification, an alkyl group can be optionally substituted.

“Alkenylene” or “alkenylene chain” refers to an unsaturated, straight or branched divalent hydrocarbon chain radical having one or more olefins and from two to twelve carbon atoms. Non-limiting examples of C₂-C₁₂ alkenylene include ethenylene, propenylene, n-butenylene, and the like. The alkenylene chain is attached to the rest of the molecule through a single bond and to a radical group (e.g., those described herein) through a single bond. The points of attachment of the alkenylene chain to the rest of the molecule and to the radical group can be through one carbon or any two carbons within the chain. Unless stated otherwise specifically in the specification, an alkenylene chain can be optionally substituted.

“Alkynyl” or “alkynyl group” refers to a straight or branched hydrocarbon chain having from two to twelve carbon atoms, and having one or more carbon-carbon triple bonds. Each alkynyl group is attached to the rest of the molecule by a single bond. Alkynyl group comprising any number of carbon atoms from 2 to 12 are included. An alkynyl group comprising up to 12 carbon atoms is a C₂-C₁₂ alkynyl, an alkynyl comprising up to 10 carbon atoms is a C₂-C₁₀ alkynyl, an alkynyl group comprising up to 6 carbon atoms is a C₂-C₆ alkynyl and an alkynyl comprising up to 5 carbon atoms is a C₂-C₅ alkynyl. A C₂-C₅ alkynyl includes C₅ alkynyls, C₄ alkynyls, C₃ alkynyls, and C₂ alkynyls. A C₂-C₆ alkynyl includes all moieties described above for C₂-C₅ alkynyls but also includes C₆ alkynyls. A C₂-C₁₀ alkynyl includes all moieties described above for C₂-C₅ alkynyls and C₂-C₆ alkynyls, but also includes C₇, C₈, C₉ and C₁₀ alkynyls. Similarly, a C₂-C₁₂ alkynyl includes all the foregoing moieties, but also includes C₁₁ and C₁₂ alkynyls. Non-limiting examples of C₂-C₁₂ alkenyl include ethynyl, propynyl, butynyl, pentynyl and the like. Unless stated otherwise specifically in the specification, an alkyl group can be optionally substituted.

“Alkynylene” or “alkynylene chain” refers to an unsaturated, straight or branched divalent hydrocarbon chain radical having one or more alkynes and from two to twelve carbon atoms. Non-limiting examples of C₂-C₁₂ alkynylene include ethynylene, propynylene, n-butynylene, and the like. The alkynylene chain is attached to the rest of the molecule through a single bond and to a radical group (e.g., those described herein) through a single bond. The points of attachment of the alkynylene chain to the rest of the molecule and to the radical group can be through any two carbons within the chain having a suitable valency. Unless stated otherwise specifically in the specification, an alkynylene chain can be optionally substituted.

“Alkoxy” refers to a group of the formula —ORₐ where Rₐ is an alkyl, alkenyl or alknyl as defined above containing one to twelve carbon atoms. Unless stated otherwise specifically in the specification, an alkoxy group can be optionally substituted.

“Aryl” refers to a hydrocarbon ring system comprising hydrogen, 6 to 18 carbon atoms and at least one aromatic ring, and which is attached to the rest of the molecule by a single bond. For purposes of this disclosure, the aryl can be a monocyclic, bicyclic, tricyclic or tetracyclic ring system, which can include fused or bridged ring systems. Aryls include, but are not limited to, aryls derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the “aryl” can be optionally substituted.

As used herein, the term “comprising” (or “comprise”, “comprises”, etc.), unless explicitly indicated otherwise or contradicted by context, has the meaning of “containing, inter alia”, i.e., “containing, among further optional elements, . . . ”. In addition thereto, this term also includes the narrower meanings of “consisting essentially of” and “consisting of”. For example, the term “A comprising B and C” has the meaning of “A containing, inter alia, B and C”, wherein A may contain further optional elements (e.g., “A containing B, C and D” would also be encompassed), but this term also includes the meaning of “A consisting essentially of B and C” and the meaning of “A consisting of B and C” (i.e., no other components than B and C are comprised in A).

The terms “effective amount” and “therapeutically effective amount” are used interchangeably in this disclosure and refer to an amount of a compound, or a salt thereof, (or pharmaceutical composition containing the compound or salt) that, when administered to a patient, is capable of performing the intended result. The “effective amount” will vary depending on the active ingredient, the state, disorder, or condition to be treated and its severity, and the age, weight, physical condition and responsiveness of the mammal to be treated.

As used herein, the terms “optional”, “optionally” and “may” denote that the indicated feature may be present but can also be absent. Whenever the term “optional”, “optionally” or “may” is used, the present invention specifically relates to both possibilities, i.e., that the corresponding feature is present or, alternatively, that the corresponding feature is absent. For example, if a component of a composition is indicated to be “optional”, the invention specifically relates to both possibilities, i.e., that the corresponding component is present (contained in the composition) or that the corresponding component is absent from the composition.

“Heteroaryl” refers to a 5 to 20 membered ring system comprising hydrogen atoms, one to nineteen carbon atoms, one to six heteroatoms selected from the group consisting of nitrogen, oxygen and sulfur, at least one aromatic ring, including compounds with aromatic resonance structures (e.g., 2-pyridone), and which is attached to the rest of the molecule by a single bond. For purposes of this disclosure, the heteroaryl can be a monocyclic, bicyclic, tricyclic or tetracyclic ring system, which can include fused or bridged ring systems; and the nitrogen, carbon or sulfur atoms in the heteroaryl can be optionally oxidized; the nitrogen atom can be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4 benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2 oxoazepinyl, oxazolyl, oxiranyl, 1-oxidopyridinyl, 1 oxidopyrimidinyl, 1-oxidopyrazinyl, 1-oxidopyridazinyl, 1 phenyl 1H pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e. thienyl). Unless stated otherwise specifically in the specification, a heteroaryl group can be optionally substituted.

“Aralkyl” or “arylalkyl” refers to a radical of the formula —R_{b}—R_{c} where R_{b} is an alkylene group as defined above and R_{c} is one or more aryl radicals as defined above, for example, benzyl, diphenylmethyl and the like. Unless stated otherwise specifically in the specification, an aralkyl group can be optionally substituted.

“Carbocyclyl,” “carbocyclic ring” or “carbocycle” refers to a rings structure, wherein the atoms which form the ring are each carbon, and which is attached to the rest of the molecule by a single bond. Carbocyclic rings can comprise from 3 to 20 carbon atoms in the ring. Carbocyclic rings include aryls and cycloalkyl, cycloalkenyl, and cycloalkynyl as defined herein. Unless stated otherwise specifically in the specification, a carbocyclyl group can be optionally substituted.

“Cycloalkyl” refers to a stable non-aromatic monocyclic or polycyclic fully saturated hydrocarbon consisting solely of carbon and hydrogen atoms, which can include fused, bridged, or spirocyclic ring systems, having from three to twenty carbon atoms (e.g., having from three to ten carbon atoms) and which is attached to the rest of the molecule by a single bond. Monocyclic cycloalkyls include, for example, cyclopropyl, cyclobutyl, cyclopentyl, cyclohexyl, cycloheptyl, and cyclooctyl. Polycyclic cycloalkyls include, for example, adamantyl, norbornyl, decalinyl, 7,7-dimethyl-bicyclo[2.2.1]heptanyl, and the like. Unless otherwise stated specifically in the specification, a cycloalkyl group can be optionally substituted.

“Cycloalkenyl” refers to a stable non-aromatic monocyclic or polycyclic hydrocarbon consisting solely of carbon and hydrogen atoms, having one or more carbon-carbon double bonds, which can include fused or bridged ring systems, having from three to twenty carbon atoms, preferably having from three to ten carbon atoms, and which is attached to the rest of the molecule by a single bond. Monocyclic cycloalkenyls include, for example, cyclopentenyl, cyclohexenyl, cycloheptenyl, cycloctenyl, and the like. Polycyclic cycloalkenyls include, for example, bicyclo[2.2.1]hept-2-enyl and the like. Unless otherwise stated specifically in the specification, a cycloalkenyl group can be optionally substituted.

“Cycloalkynyl” refers to a stable non-aromatic monocyclic or polycyclic hydrocarbon consisting solely of carbon and hydrogen atoms, having one or more carbon-carbon triple bonds, which can include fused or bridged ring systems, having from three to twenty carbon atoms, preferably having from three to ten carbon atoms, and which is attached to the rest of the molecule by a single bond. Monocyclic cycloalkynyl include, for example, cycloheptynyl, cyclooctynyl, and the like. Unless otherwise stated specifically in the specification, a cycloalkynyl group can be optionally substituted.

“Haloalkyl” refers to an alkyl, as defined above, that is substituted by one or more halo radicals, e.g., trifluoromethyl, difluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a haloalkyl group can be optionally substituted.

“Heterocyclyl,” “heterocyclic ring” or “heterocycle” refers to a stable saturated, unsaturated, or aromatic 3- to 20-membered ring which consists of two to nineteen carbon atoms and from one to six heteroatoms selected from the group consisting of nitrogen, oxygen and sulfur, and which is attached to the rest of the molecule by a single bond. Heterocyclyl or heterocyclic rings include heteroaryls, heterocyclylalkyls, heterocyclylalkenyls, and hetercyclylalkynyls. Unless stated otherwise specifically in the specification, the heterocyclyl can be a monocyclic, bicyclic, tricyclic or tetracyclic ring system, which can include fused, bridged, or spirocyclic ring systems; and the nitrogen, carbon or sulfur atoms in the heterocyclyl can be optionally oxidized; the nitrogen atom can be optionally quaternized; and the heterocyclyl can be partially or fully saturated. Examples of such heterocyclyl include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2 oxopiperazinyl, 2 oxopiperidinyl, 2 oxopyrrolidinyl, oxazolidinyl, piperidinyl, piperazinyl, 4 piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1 oxo thiomorpholinyl, and 1,1 dioxo thiomorpholinyl. Unless stated otherwise specifically in the specification, a heterocyclyl group can be optionally substituted.

The term “protective gas”, as used herein, refers to an inert gas, preferably argon. In other embodiments, also a different protective gas can be employed, e.g., elementary gases such as nitrogen, noble gases such as helium, neon, argon, krypton, xenon, and gaseous molecular compounds like sulfur hexafluoride.

The term “substituted” used herein means any of the groups described herein (e.g., alkyl, alkoxy, aryl, heterocyclyl, and/or heteroaryl) wherein at least one hydrogen atom is replaced by a bond to a non-hydrogen atom such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also means any of the above groups in which one or more hydrogen atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more hydrogen atoms are replaced with —NR_{g}Rₕ, —NR_{g}C(═O)Rₕ, —NR_{g}C(═O)NR_{g}Rₕ, —NR_{g}C(═O)ORₕ, —NR_{g}SO₂Rₕ, —OC(═O)NR_{g}Rₕ, —OR_{g}, —SR_{g}, —SORₕ, —SO₂R_{g}, —OSO₂R_{g}, —SO₂OR_{g}, ═NSO₂R_{g}, and —SO₂NR_{g}Rₕ. “Substituted” also means any of the above groups in which one or more hydrogen atoms are replaced with —C(═O)R_{g}, —C(═O)OR_{g}, —C(═O)NR_{g}Rₕ, —CH₂SO₂R_{g}, —CH₂SO₂NR_{g}Rₕ. In the foregoing, R_{g} and Rₕ are the same or different and independently hydrogen, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl. “Substituted” further means any of the above groups in which one or more hydrogen atoms are replaced by a bond to an amino, cyano, hydroxyl, imino, nitro, oxo, thioxo, halo, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl group. In addition, each of the foregoing substituents can also be optionally substituted with one or more of the above substituents.

As used herein, the term “treatment” (or “treating”) in relation to a disease or disorder refers to the management and care of a patient for the purpose of combating the disease or disorder, such as to reverse, alleviate, inhibit or delay the disease or disorder, or one or more symptoms of such disease or disorder. It also refers to the administration of a compound or a composition for the purpose of preventing the onset of symptoms of the disease or disorder, alleviating such symptoms, or eliminating the disease or disorder. Preferably, the “treatment” is curative, ameliorating or palliative.

It is to be understood that wherever numerical ranges are provided/disclosed herein, all values and subranges encompassed by the respective numerical range are meant to be encompassed within the scope of the invention. Accordingly, the present invention specifically and individually relates to each value that falls within a numerical range disclosed herein, as well as each subrange encompassed by a numerical range disclosed herein.

It is further to be understood that the present invention specifically relates to each and every combination of features and embodiments described herein, including any combination of general and/or preferred features/embodiments. In particular, the invention specifically relates to each combination of meanings (including general and/or preferred meanings) for the various groups and variables comprised in formula (I).

### Compounds of the Present Disclosure

The present invention provides a novel group of active compounds based on the psychoactive compound psilocin. The psilocin derivatives provided herein exhibit improved pharmacokinetic properties during uptake as compared to psilocin, as well as reduced side effects resulting from the metabolites thus formed. Due to the affinity of the novel psilocin derivatives for the 5-HT_{2A}-receptor, these derivatives are particularly advantageous for use in therapy, e.g., in the treatment of depression or drug addiction.

In one aspect, the present invention provides a novel psilocin derivative according to the following general formula (I):

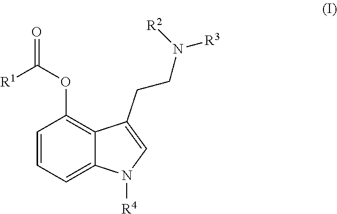

wherein R¹ is selected from the group consisting of —O—(C₁₋₁₂ alkyl), —O-heteroaryl, —O—CH₂-aryl, heterocyclyl, —CH(—NH₂)-(heteroaryl), —O-(alkylene)-O-alkyl and —CH(—NH₂)-alkyl, wherein the alkyl, alkylene, aryl, heteroaryl and heterocyclyl groups are each optionally substituted with one or more substituents, wherein when R² and R³ are methyl, R¹ is not —CH₂—NH₂ or —CH(—NH₂)—CH₃;
R² and R³ are each independently selected from hydrogen, methyl and ethyl, provided that R² and R³ are not both hydrogen; and
R⁴ is hydrogen or —C(═O)—O—(C₁₋₆ alkyl), or a pharmaceutically acceptable salt thereof.

In some embodiments, the alkyl, alkylene, aryl, heteroaryl and heterocyclyl groups in the group R¹ are optionally substituted with one or more substituents selected from the group consisting of halogen, aryl, amino, heteroaryl alkoxy, thioalkoxy, hydroxyl, thiol, amino, guanidino, —C(═O)—NR_{A}R_{B}, —C(═O)—OR_{A}, and disulfanyl, and R_{A} and R_{B} are independently selected from the group consisting of hydrogen and alkyl.

In some embodiments of the compound of Formula (I), R¹ is selected from the group consisting of —O—(C₁₋₁₂ alkyl), —O—CH₂-phenyl, —CH₂—NH₂, —CH(—NH₂)—CH₃, —CH(—NH₂)—CH(—CH₃)—CH₃, —CH(—NH₂)—CH₂—CH(—CH₃)—CH₃, —CH(—NH₂)—CH(—CH₃)—CH₂CH₃, —CH(—NH₂)—CH₂CH₂—S—CH₃, —CH(—NH₂)—CH₂—SH, —CH(—NH₂)—CH₂—OH, —CH(—NH₂)—CH(—CH₃)—OH, —CH(—NH₂)—CH₂—C(═O)—NH₂, —CH(—NH₂)—CH₂CH₂—C(═O)—NH₂, —CH(—NH₂)—CH₂—COOH, —CH(—NH₂)—CH₂CH₂—COOH, —CH(—NH₂)—CH₂CH₂CH₂CH₂—NH₂, —CH(—NH₂)—CH₂CH₂CH₂—NH—C(═NH)—NH₂, —CH(—NH₂)—CH₂-(1H-imidazol-4-yl), —CH(—NH₂)—CH₂-phenyl, —CH(—NH₂)—CH₂-(4-hydroxyphenyl), —CH(—NH₂)—CH₂-(1H-indol-3-yl), -(pyrrolidin-2-yl), -(4-hydroxypyrrolidin-2-yl), —CH(—NH₂)—CH₂—S—S—CH₂—CH(—NH₂)—COOH, —CH(—NH₂)—CH₂CH₂CH₂—NH₂, —CH(—NH₂)—CH₂CH₂CH₂—NH—C(═O)—NH₂, —CH₂—NH—CH₃, —CH(—NH₂)—CH₂CH₂—SH, —CH(—NH₂)—CH₂CH₂—OH, —CH(—NH₂)—CH₂-(3,4-dihydroxyphenyl), —CH(—NH₂)—CH₂-(5-hydroxy-1H-indol-3-yl), —CH₂CH₂—NH₂, —CH₂CH₂CH₂—NH₂, —CH(—CH₃)—CH₂—NH₂, —C(—NH₂)═CH₂, —O-(1-[R⁴]-3-[(-CH₂CH₂—N(—R²)—R³)]-1H-indol-4-yl), —O—(C₁₋₁₂ alkylene)-O-(1-[R⁴]-3-[(-CH₂CH₂—N(—R²)—R³)]-1H-indol-4-yl), —CH(—NH₂)—CH₂—COO-(1-[R⁴]-3-[(-CH₂CH₂—N(—R²)—R³)]-1H-indol-4-yl), —CH(—NH₂)—CH₂CH₂—COO-(1-[R⁴]-3-[(-CH₂CH₂—N(—R²)—R³)]-1H-indol-4-yl), —CH(—NH₂)—CH₂—S—S—CH₂—CH(—NH₂)—COO-(1-[R⁴]-3-[(-CH₂CH₂—N(—R²)—R³)]-1H-indol-4-yl), —O-(5-(aminomethyl)isoxazol-3-yl), and —CH(—NH₂)-(3-hydroxy-isoxazol-5-yl);
R² and R³ are each independently selected from hydrogen, methyl and ethyl, provided that R² and R³ are not both hydrogen; and
R⁴ is hydrogen or —C(═O)—O—(C₁₋₆ alkyl), or a pharmaceutically acceptable salt thereof.

In some embodiments, R¹ is selected from the group consisting of —O—(C₁₋₁₂ alkyl) and —O—CH₂-phenyl. The —O—(C₁₋₁₂ alkyl) group may be, for example, a —O—(C₂₋₅ alkyl) group, such as, e.g., ethoxy, n-propoxy, isopropoxy, n-butyloxy, isobutyloxy, tert-butyloxy, or neopentyloxy. Yet, R¹ may also be, for example, a —O—(C₆₋₁₂ alkyl) (e.g., a C₆ alkoxy, a C₇ alkoxy, a C₈ alkoxy, a C₉ alkoxy, a C₁₀ alkoxy, a C₁₁ alkoxy, or a C₁₂ alkoxy).

In some embodiments, R¹ is selected from the group consisting of —CH₂—NH₂, —CH(—NH₂)—CH₃, —CH(—NH₂)—CH(—CH₃)—CH₃, —CH(—NH₂)—CH₂—CH(—CH₃)—CH₃, —CH(—NH₂)—CH(—CH₃)—CH₂CH₃, —CH(—NH₂)—CH₂CH₂—S—CH₃, —CH(—NH₂)—CH₂—SH, —CH(—NH₂)—CH₂—OH, —CH(—NH₂)—CH(—CH₃)—OH, —CH(—NH₂)—CH₂—C(═O)—NH₂, —CH(—NH₂)—CH₂CH₂—C(═O)—NH₂, —CH(—NH₂)—CH₂—COOH, —CH(—NH₂)—CH₂CH₂—COOH, —CH(—NH₂)—CH₂CH₂CH₂CH₂—NH₂, —CH(—NH₂)—CH₂CH₂CH₂—NH—C(═NH)—NH₂, —CH(—NH₂)—CH₂-(1H-imidazol-4-yl), —CH(—NH₂)—CH₂-phenyl, —CH(—NH₂)—CH₂-(4-hydroxyphenyl), —CH(—NH₂)—CH₂-(1H-indol-3-yl), and -(pyrrolidin-2-yl).

In some embodiments, R¹ is selected from the group consisting of —CH(—NH₂)—CH(—CH₃)—CH₃, —CH(—NH₂)—CH₂—CH(—CH₃)—CH₃, —CH(—NH₂)—CH(—CH₃)—CH₂CH₃, —CH(—NH₂)—CH₂CH₂—S—CH₃, —CH(—NH₂)—CH₂—SH, —CH(—NH₂)—CH₂—OH, —CH(—NH₂)—CH(—CH₃)—OH, —CH(—NH₂)—CH₂—C(═O)—NH₂, —CH(—NH₂)—CH₂CH₂—C(═O)—NH₂, —CH(—NH₂)—CH₂—COOH, —CH(—NH₂)—CH₂CH₂—COOH, —CH(—NH₂)—CH₂CH₂CH₂CH₂—NH₂, —CH(—NH₂)—CH₂CH₂CH₂—NH—C(═NH)—NH₂, —CH(—NH₂)—CH₂-(1H-imidazol-4-yl), —CH(—NH₂)—CH₂-phenyl, —CH(—NH₂)—CH₂-(4-hydroxyphenyl), —CH(—NH₂)—CH₂-(1H-indol-3-yl), and -(pyrrolidin-2-yl).

In some embodiments, R¹ is selected from the group consisting of -(4-hydroxypyrrolidin-2-yl), —CH(—NH₂)—CH₂—S—S—CH₂—CH(—NH₂)—COOH, —CH(—NH₂)—CH₂CH₂CH₂—NH₂, —CH(—NH₂)—CH₂CH₂CH₂—NH—C(═O)—NH₂, —CH₂—NH—CH₃, —CH(—NH₂)—CH₂CH₂—SH, —CH(—NH₂)—CH₂CH₂—OH, —CH(—NH₂)—CH₂-(3,4-dihydroxyphenyl), —CH(—NH₂)—CH₂-(5-hydroxy-1H-indol-3-yl), —CH₂CH₂—NH₂, —CH₂CH₂CH₂—NH₂, —CH(—CH₃)—CH₂—NH₂, and —C(—NH₂)═CH₂.

In some embodiments, R² and R³ are methyl. In some embodiments, R² and R³ are ethyl. In some embodiments, R² is methyl and R³ is hydrogen. In some embodiments, R² is ethyl and R³ is hydrogen. Preferably, R² and R³ are each methyl.

In some embodiments, R⁴ is hydrogen. In some embodiments, R⁴ is —C(═O)—O—(C₂₋₄ alkyl). Preferably, R⁴ is hydrogen.

In a preferred aspect, the novel psilocin derivative according to formula (I) is a compound having the following formula or a pharmaceutically acceptable salt thereof:

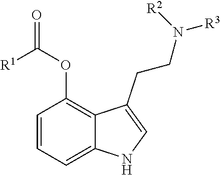

wherein, R¹ is selected from the group consisting of —O—(C₂₋₅ alkyl), —O—CH₂-phenyl, —CH₂—NH₂, —CH(—NH₂)—CH₂—COOH, and —CH(—NH₂)—CH₂(1H-indol-3-yl). In preferred embodiments, R¹ is selected from the group consisting of —O—CH₂CH₃, —O—CH₂CH₂CH₃, —O—CH(—CH₃)—CH₃, —O—CH₂CH₂CH₂CH₃, —O—CH₂—CH(—CH₃)—CH₃, —O—C(—CH₃)₃, —O—CH₂—C(—CH₃)₃, —O—CH₂-phenyl (i.e., benzyloxy), —CH₂—NH₂, —CH(—NH₂)—CH₂—COOH, and —CH(—NH₂)—CH₂(1H-indol-3-yl).

In some embodiments, R² is methyl or ethyl. Preferably, R² is methyl.

In some embodiments, R³ is methyl or ethyl. Preferably, R³ is methyl.

It is particularly preferred that R² and R³ are each methyl.

Preferred examples of the novel psilocin derivatives according to the invention include any one of the following compounds (as well as pharmaceutically acceptable salts of any of these compounds):

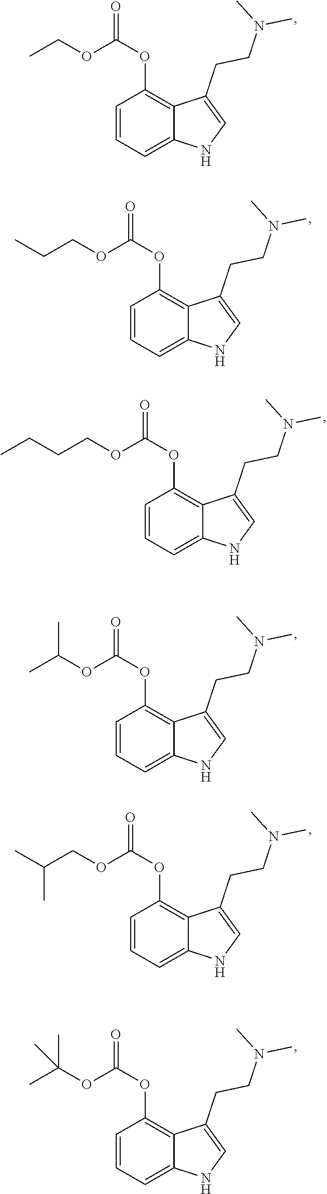

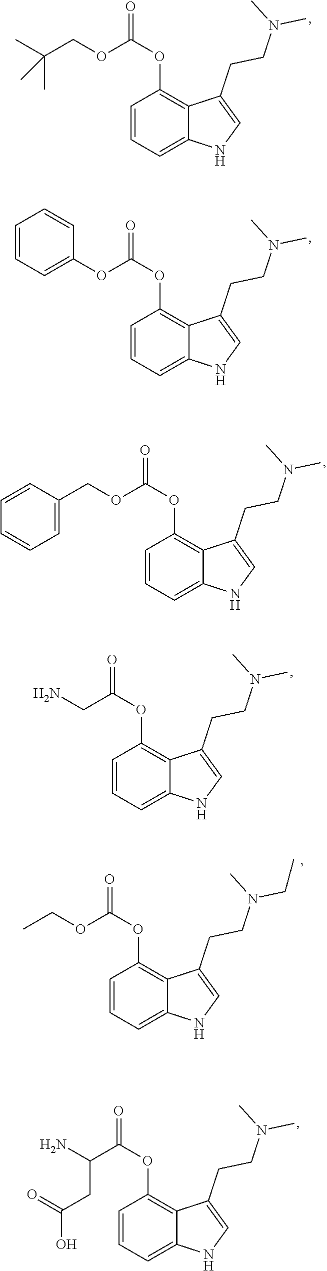

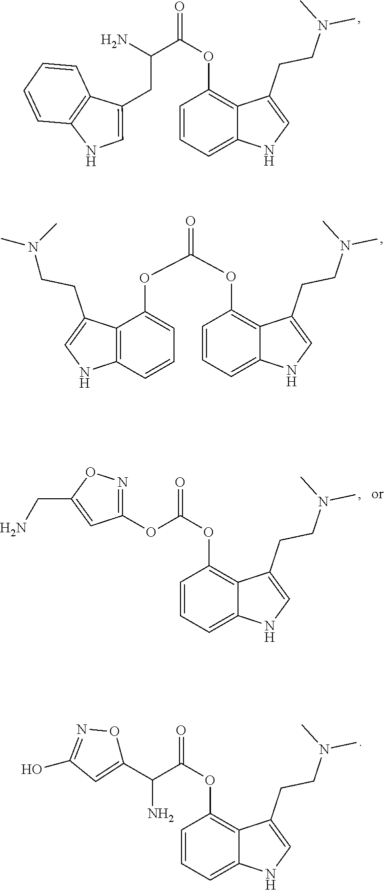

In some embodiments, the present invention provides the compounds having the following molecular structures:

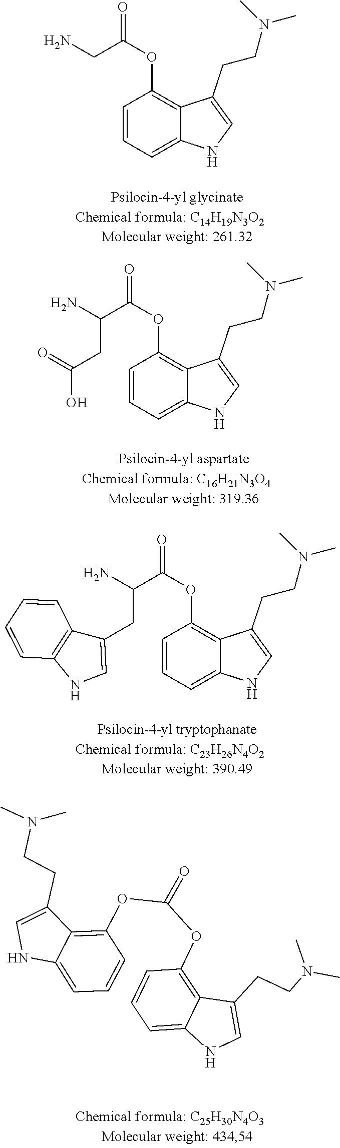

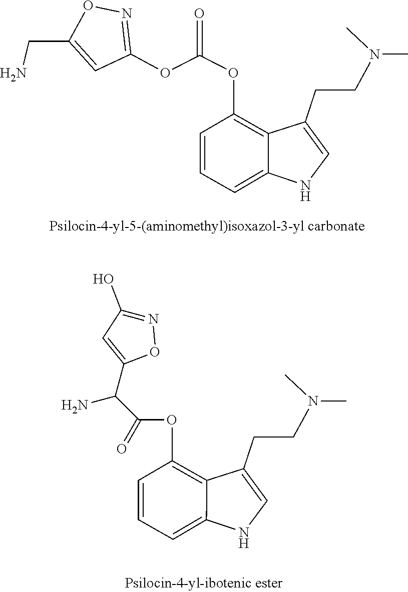 [SMILES: CN(C)CCc1c[nH]c2cccc(OC(=O)Oc3cc(CN)on3)c12 ; CN(C)CCc1c[nH]c2cccc(OC(=O)C(N)c3cc(O)no3)c12]

In some embodiments, the invention provides psilocin derivatives having the following structures:

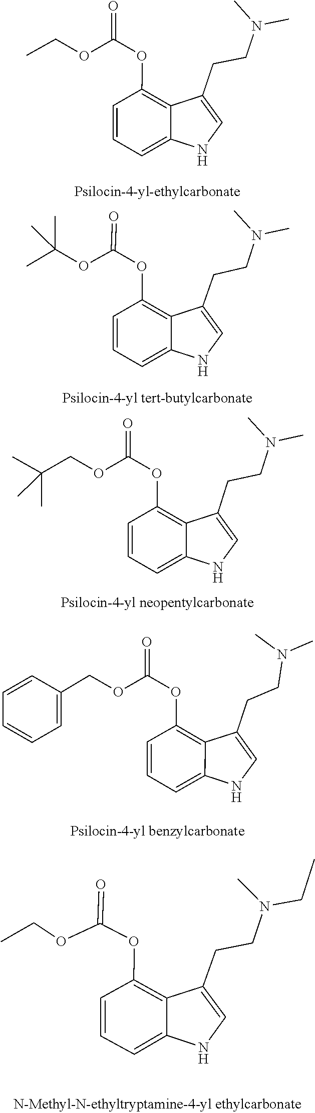

The present invention relates to the psilocin derivatives described herein in any form, e.g., in non-salt form or in the form of a salt, particularly a pharmaceutically acceptable salt.

The scope of the present invention thus embraces all pharmaceutically acceptable salt forms of the psilocin derivatives of formula (I) which may be formed, e.g., by protonation of an atom carrying an electron lone pair which is susceptible to protonation, such as an amino group, with an inorganic or organic acid, or as a salt of an acid group (such as a carboxylic acid group) with a physiologically acceptable cation. Exemplary base addition salts comprise, for example: alkali metal salts such as sodium or potassium salts; alkaline earth metal salts such as calcium or magnesium salts; zinc salts; ammonium salts; aliphatic amine salts such as trimethylamine, triethylamine, dicyclohexylamine, ethanolamine, diethanolamine, triethanolamine, procaine salts, meglumine salts, ethylenediamine salts, or choline salts; aralkyl amine salts such as N,N-dibenzylethylenediamine salts, benzathine salts, benethamine salts; heterocyclic aromatic amine salts such as pyridine salts, picoline salts, quinoline salts or isoquinoline salts; quaternary ammonium salts such as tetramethylammonium salts, tetraethylammonium salts, benzyltrimethylammonium salts, benzyltriethylammonium salts, benzyltributylammonium salts, methyltrioctylammonium salts or tetrabutylammonium salts; and basic amino acid salts such as arginine salts, lysine salts, or histidine salts. Exemplary acid addition salts comprise, for example: mineral acid salts such as hydrochloride, hydrobromide, hydroiodide, sulfate salts (such as, e.g., sulfate or hydrogensulfate salts), nitrate salts, phosphate salts (such as, e.g., phosphate, hydrogenphosphate, or dihydrogenphosphate salts), carbonate salts, hydrogencarbonate salts, perchlorate salts, borate salts, or thiocyanate salts; organic acid salts such as acetate, propionate, butyrate, pentanoate, hexanoate, heptanoate, octanoate, cyclopentanepropionate, decanoate, undecanoate, oleate, stearate, lactate, maleate, oxalate, fumarate, tartrate, malate, citrate, succinate, adipate, gluconate, glycolate, nicotinate, benzoate, salicylate, ascorbate, pamoate (embonate), camphorate, glucoheptanoate, or pivalate salts; sulfonate salts such as methanesulfonate (mesylate), ethanesulfonate (esylate), 2-hydroxyethanesulfonate (isethionate), benzenesulfonate (besylate), p-toluenesulfonate (tosylate), 2-naphthalenesulfonate (napsylate), 3-phenylsulfonate, or camphorsulfonate salts; glycerophosphate salts; and acidic amino acid salts such as aspartate or glutamate salts.

Further pharmaceutically acceptable salts are described in the literature, e.g., in Stahl P H & Wermuth C G (eds.), “Handbook of Pharmaceutical Salts: Properties, Selection, and Use”, Wiley-VCH, 2002 and in the references cited therein. Preferred examples of a pharmaceutically acceptable salt of the psilocin derivatives according to the invention include, e.g., a fumarate salt, a maleate salt, an oxalate salt, a malate salt, a tartrate salt, or a methanesulfonate (mesylate) salt. A particularly preferred pharmaceutically acceptable salt is a fumarate salt. A further particularly preferred pharmaceutically acceptable salt is an oxalate salt.

The scope of the present invention also embraces the psilocin derivatives provided herein in any hydrated or solvated form, and in any physical form, including any amorphous or crystalline forms.

Moreover, the psilocin derivatives of formula (I) may exist in the form of different isomers, in particular stereoisomers (e.g., enantiomers or diastereomers). All such isomers of the compounds of formula (I) are contemplated as being part of the present invention, either in admixture or in pure or substantially pure form. Any tautomers of the compounds described herein are also embraced by the present invention. As for stereoisomers, the invention embraces the isolated optical isomers of the psilocin derivatives according to the invention as well as any mixtures thereof (including, in particular, racemic mixtures/racemates). The racemates can be resolved by physical methods, such as, e.g., fractional crystallization, separation or crystallization of diastereomeric derivatives, or separation by chiral column chromatography. The individual optical isomers may also be prepared by using corresponding optically active starting materials in their synthesis, or they may be obtained from corresponding racemates via salt formation with an optically active acid followed by crystallization. In the case of the compounds psilocin-4-yl aspartate and psilocin-4-yl tryptophanate, while the carbon atom carrying the —NH₂ group (corresponding to the C_{α}-atom of the respective amino acid aspartate or tryptophan) may be present in the (S)-configuration, in the (R)-configuration, or as a racemic mixture, it is preferred that said carbon atom is present in the (S)-configuration (as in the naturally occurring amino acids L-aspartate and L-tryptophan). For any other compounds of formula (I) having an amino acid residue as R¹, the C_{α}-atom of the respective amino acid residue may likewise be present in the (S)-configuration, in the (R)-configuration, or as a racemic mixture, whereby it is preferred that said C_{α}-atom is present in the (S)-configuration.

The scope of the invention also embraces psilocin derivatives of formula (I), in which one or more atoms are replaced by a specific isotope of the corresponding atom. For example, the invention encompasses compounds of formula (I), in which one or more hydrogen atoms (or, e.g., all hydrogen atoms) are replaced by deuterium atoms (i.e., ²H; also referred to as “D”). Accordingly, the invention also embraces compounds of formula (I) which are enriched in deuterium. Naturally occurring hydrogen is an isotopic mixture comprising about 99.98 mol-% hydrogen-1 (¹H) and about 0.0156 mol-% deuterium (²H or D). The content of deuterium in one or more hydrogen positions in the compounds of formula (I) can be increased using deuteration techniques known in the art. For example, a compound of formula (I) or a reactant or precursor to be used in the synthesis of the compound of formula (I) can be subjected to an H/D exchange reaction using, e.g., heavy water (D₂O). The content of deuterium can be determined, e.g., using mass spectrometry or NMR spectroscopy. It is generally preferred that the psilocin derivatives of formula (I) are not enriched in deuterium. Accordingly, the presence of naturally occurring hydrogen atoms or ¹H hydrogen atoms in the compounds of formula (I) is preferred. The invention thus particularly relates to a psilocin derivative of formula (I) in which all hydrogen atoms are naturally occurring hydrogen atoms or ¹H hydrogen atoms.

Due to their molecular structure, the psilocin derivatives according to the invention allow an improved method of production and, furthermore, exhibit novel beneficial pharmacological properties.

In particular, due to their specific molecular structure, the adducts of psilocin according to the invention are pharmacologically released, taken up and metabolized in the human body with different pharmacokinetics (as compared to psilocybin).

The pharmacological “inactivation” of the active compound (psilocin) in the form of a prodrug reduces the potential for abuse because a rapid “flooding” of the active compound is suppressed.

The potential for addiction of psychotropic substances is related to a rapid increase of their concentration upon uptake. Therefore, active compounds leading only to a slow increase from the initial concentration are sought from a pharmaceutical point of view.

The present invention provides compounds that have been found to act more rapidly than psilocybin, e.g. as they are hydrolyzed to psilocin inside the body faster than psilocybin is hydrolyzed, which makes these compounds particularly suitable as fast-acting therapeutic drugs. Moreover, the compounds provided herein exert their effect on the organism only after endogenous metabolization into the actual active compound psilocin, whereby a longer-lasting effect (depot effect) is obtained. Compounds that are hydrolyzed more slowly can provide a particularly long depot effect. The invention thus allows to finetune the release properties of the psilocin derivatives provided herein, particularly by choosing a more or less rapidly hydrolysable group as R¹ in formula (I).

The steadier and more uniform release of the active compound in the organism furthermore contributes to reducing side effects.

The “depot effect” resulting from such delayed release is therefore a particular advantage of the present invention.

In further embodiments, by selection of the amino acid derivatives to be used, a beneficial additional pharmacological effect of the psilocin derivative, besides the retarding effect, can be obtained.

Exemplary amino acid derivatives have been described above (e.g., psilocin derivatives wherein R¹ is —CH₂—NH₂, corresponding to a glycine derivative, wherein R¹ is —CH(—NH₂)—CH₂—COOH, corresponding to an aspartate derivative, or wherein R¹ is —CH(—NH₂)—CH₂-(1H-indol-3-yl), corresponding to a tryptophan derivative).

Thus, for example, in the case of psilocin aspartate, the resulting betaine structure provides for better uptake of the psilocin aspartate. In the case of psilocin tryptophanate, the amino acid tryptophan, which is released by metabolization of psilocin tryptophanate, reduces or mitigates the side effect of “serotonin starvation” which may occur in the course of conventional psilocin therapy.

Further aspects of the present invention relate to methods of producing the novel psilocin derivatives provided herein as well as methods and uses, particularly methods of treatment and therapeutic uses, of these novel compounds.

### Methods of Making the Compounds of the Present Disclosure

In one aspect, the present disclosure provides methods of making the compounds of the present disclosure.

In some embodiments, the present disclosure provides a method for producing a psilocin derivative (as described herein), comprising the steps of:
(a) preparing a suspension of psilocin in a solvent I;
(b) adding an activating agent under a protective gas atmosphere;
(c) adding a derivatization agent;
(d) stirring the mixture under a protective gas atmosphere (e.g., for at least 3 hours);
(e) stopping the reaction by dilution with solvent (e.g., solvent I from step (a));
(f) concentrating the solvent;
(g) dissolving the residue in a solvent II;
(h) extracting with 1 M HCl, water and saturated saline solution;
(i) drying the organic phase over a desiccant at 40-60° C. under vacuum (or reduced pressure);
(j) obtaining the crude product;
(k) purifying the crude product by recrystallization and/or column chromatography;
(l) obtaining the psilocin derivative according to the invention.

In one embodiment, in step (a), between 0.21 mmol and 2.1 mmol of psilocin are suspended in 10 ml to 100 ml of solvent I, wherein solvent I is selected from tetrahydrofuran, dioxane, 2-methyltetrahydrofuran and dichloromethane.

This can be done at a temperature between −78° C. and 45° C., preferably at a temperature between 5° C. and 40° C., more preferably at room temperature (293.15 Kelvin; 20° C.).

In one embodiment, in step (b), between 0.5 mmol and 5 mmol of an activating agent are added, such as, e.g., a nitrogen base and/or a carbodiimide.

In this case, in preferred embodiments, the nitrogen base is selected from triethylamine, diisopropyl ethylamine, pyridine, and 4-dimethyl aminopyridine. The carbodiimide, which may be added, is preferably selected from dicyclohexyl carbodiimide (DCC), diisopropyl carbodiimide (DIC), and 1-ethyl-3-(3-dimethylaminopropyl) carbodiimide (EDC).

The solution obtained is aerated with protective gas.

It is also possible to use a deprotonating agent such as n-butyllithium (n-BuLi), and/or an acid anhydride or an acid chloride.

In one embodiment, in step (c), between 0.25 mmol and 2.5 mmol of a derivatization agent are added dropwise through a septum, wherein the derivatization agent is selected from ethyl chloroformate, di-tert-butyl pyrocarbonate, N-carbobenzoxy-glycine, N-(9-fluorenylmethyloxycarbonyl)-L-tryptophan, and 4-benzyl N-carbobenzoxy-L-aspartate.

In one embodiment, in step (d), the mixture is stirred between 2 and 10 hours at 20-28° C. under protective gas atmosphere. In one embodiment, it is stirred for at least 3 hours and up to 6 hours; and/or at 20° C. under protective gas atmosphere.

In a further embodiment, in step (e), the reaction is stopped by adding between 30 ml and 300 ml of the solvent I from step (a).

In a further embodiment, in step (f), the mixture is dried, preferably in a rotatory evaporator under vacuum, and is redissolved in 30 ml to 300 ml of solvent II, wherein solvent II is selected from ethyl acetate, diethyl ether, and dichloromethane.

In one embodiment, in step (h), extraction is performed with between 20 ml and 200 ml of 1 molar (1 M) hydrochloric acid. In one embodiment, subsequent extraction with between 20 ml and 200 ml water is performed. In one embodiment, subsequent extraction with between 20 ml and 200 ml saturated saline solution is performed.

In a further embodiment, in step (i), the mixture is dried. Particularly preferred is drying with a desiccant at a temperature between 35° C. and 60° C. and a vacuum (reduced pressure) of 30-60 mbar.

Preferred desiccants are anhydrous calcium chloride, anhydrous sodium carbonate, anhydrous potassium carbonate, anhydrous sodium sulfate, anhydrous magnesium sulphate, or anhydrous calcium sulfate. In one embodiment, the desiccant is anhydrous MgSO₄, the temperature is 45° C., and the vacuum is 40 mbar.

The crude product obtained in steps (a) to (j) contains the psilocin derivative according to the invention.

In a further embodiment, the crude product is further purified. The purification can be conducted, e.g., by dissolving in isopropanol with subsequent evaporation at 50° C. and 400 mbar until crystallization and/or column purification over 50 g silica using the eluent mixture dichloromethane/methanol, e.g., in a ratio of 8:2 in one embodiment. Other column materials and eluents are known in the art can also be used.

In one embodiment, in the recrystallization from isopropanol, a strengthening or intensification of the crystallization is facilitated by addition of diisopropyl ether.

With this method, yields of more than 65 wt-% (gravimetric determination of the amount of the end product, relative to the initial materials) are achieved. In some embodiments, yields of more than 70 wt-%, more than 75 wt-%, more than 80 wt-%, and up to 85 wt-%, up to 90 wt-%, and even up to 95 wt-% are achieved.

Further details on the method of production are provided in the examples and will be apparent to the person skilled in the art.

The present invention thus provides compounds having the general molecular structure (I), which can be produced in high purity using the method according to the invention:

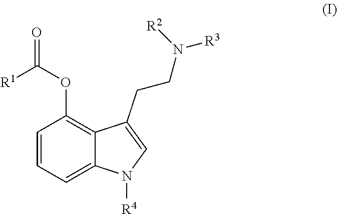

wherein the groups in formula (I) are defined as follows:
R¹ is selected from —O—(C₁₋₁₂ alkyl), —O—CH₂-phenyl, —CH₂—NH₂, —CH(—NH₂)—CH₃, —CH(—NH₂)—CH(—CH₃)—CH₃, —CH(—NH₂)—CH₂—CH(—CH₃)—CH₃, —CH(—NH₂)—CH(—CH₃)—CH₂CH₃, —CH(—NH₂)—CH₂CH₂—S—CH₃, —CH(—NH₂)—CH₂—SH, —CH(—NH₂)—CH₂—OH, —CH(—NH₂)—CH(—CH₃)—OH, —CH(—NH₂)—CH₂—C(═O)—NH₂, —CH(—NH₂)—CH₂CH₂—C(═O)—NH₂, —CH(—NH₂)—CH₂—COOH, —CH(—NH₂)—CH₂CH₂—COOH, —CH(—NH₂)—CH₂CH₂CH₂CH₂—NH₂, —CH(—NH₂)—CH₂CH₂CH₂—NH—C(═NH)—NH₂, —CH(—NH₂)—CH₂-(1H-imidazol-4-yl), —CH(—NH₂)—CH₂-phenyl, —CH(—NH₂)—CH₂-(4-hydroxyphenyl), —CH(—NH₂)—CH₂-(1H-indol-3-yl), -(pyrrolidin-2-yl), -(4-hydroxypyrrolidin-2-yl), —CH(—NH₂)—CH₂—S—S—CH₂—CH(—NH₂)—COOH, —CH(—NH₂)—CH₂CH₂CH₂—NH₂, —CH(—NH₂)—CH₂CH₂CH₂—NH—C(═O)—NH₂, —CH₂—NH—CH₃, —CH(—NH₂)—CH₂CH₂—SH, —CH(—NH₂)—CH₂CH₂—OH, —CH(—NH₂)—CH₂-(3,4-dihydroxyphenyl), —CH(—NH₂)—CH₂-(5-hydroxy-1H-indol-3-yl), —CH₂CH₂—NH₂, —CH₂CH₂CH₂—NH₂, —CH(—CH₃)—CH₂—NH₂, —C(—NH₂)═CH₂, —O-(1-[R⁴]-3-[(-CH₂CH₂—N(—R²)—R³)]-1H-indol-4-yl), —O—(C₁₋₁₂ alkylene)-O-(1-[R⁴]-3-[(-CH₂CH₂—N(—R²)—R³)]-1H-indol-4-yl), —CH(—NH₂)—CH₂—COO-(1-[R⁴]-3-[(-CH₂CH₂—N(—R²)—R³)]-1H-indol-4-yl), —CH(—NH₂)—CH₂CH₂—COO-(1-[R⁴]-3-[(-CH₂CH₂—N(—R²)—R³)]-1H-indol-4-yl), —CH(—NH₂)—CH₂—S—S—CH₂—CH(—NH₂)—COO-(1-[R⁴]-3-[(-CH₂CH₂—N(—R²)—R³)]-1H-indol-4-yl), —O-(5-(aminomethyl)isoxazol-3-yl), and —CH(—NH₂)-(3-hydroxy-isoxazol-5-yl).
R² and R³ are each independently selected from hydrogen, methyl and ethyl, provided that R² and R³ are not both hydrogen.
R⁴ is hydrogen or —C(═O)—O—(C₁₋₆ alkyl).

In particular, the present invention provides compounds having the following general molecular structure, which can likewise be produced in high purity using the method according to the invention:

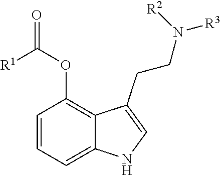

wherein the groups in this formula are defined as follows:
R¹ is —O—(C₂₋₅ alkyl), particularly ethoxy, n-propoxy, isopropoxy, n-butyloxy, isobutyloxy, tert-butyloxy, or neopentyloxy.
R² is methyl (—CH₃) or ethyl (—C₂H₅), particularly methyl.
R³ is methyl (—CH₃) or ethyl (—C₂H₅), particularly methyl.

### Pharmaceutical Compositions

The present invention provides a pharmaceutical/pharmacological composition comprising at least one psilocin derivative according to the invention and optionally one or more pharmaceutically acceptable excipients. The invention likewise relates to the psilocin derivatives provided herein, or the aforementioned pharmaceutical composition, for use in therapy (or for use as a medicament).

The psilocin derivatives provided herein may be administered as compounds per se or may be formulated as pharmaceutical/pharmacological compositions or medicaments. The pharmaceutical compositions/medicaments may optionally comprise one or more pharmaceutically acceptable excipients, such as carriers, diluents, fillers, disintegrants, lubricating agents, binders, colorants, pigments, stabilizers, preservatives, and/or antioxidants.

The pharmaceutical compositions can be formulated by techniques known to the person skilled in the art, such as the techniques published in “Remington: The Science and Practice of Pharmacy”, Pharmaceutical Press, 22^{nd} edition. The pharmaceutical compositions can be formulated as dosage forms for oral, parenteral, such as intramuscular, intravenous, subcutaneous, intradermal, intraarterial, intracardial, rectal, nasal, topical, aerosol or vaginal administration. Dosage forms for oral administration include coated and uncoated tablets, soft gelatin capsules, hard gelatin capsules, lozenges, troches, solutions, emulsions, suspensions, syrups, elixirs, powders and granules for reconstitution, dispersible powders and granules, medicated gums, chewing tablets and effervescent tablets. Dosage forms for parenteral administration include solutions, emulsions, suspensions, dispersions and powders and granules for reconstitution. Emulsions are a preferred dosage form for parenteral administration. Dosage forms for rectal and vaginal administration include suppositories and ovula. Dosage forms for nasal administration can be administered via inhalation and insufflation, for example by a metered inhaler. Dosage forms for topical administration include creams, gels, ointments, salves, patches and transdermal delivery systems.

### Methods of Treatment

The invention further relates to a psilocin derivative as described herein (which may be present in non-salt form or in the form of a pharmaceutically acceptable salt), or a pharmaceutical composition comprising at least one psilocin derivative, for use in the treatment of a serotonin 5-HT_{2A} receptor associated disease/disorder. In particular, the invention relates to a psilocin derivative or a pharmaceutical composition, as described herein, for use in the treatment of an anxiety disorder, attention deficit hyperactivity disorder (ADHD), depression, cluster headache, a condition associated with cancer, diminished drive, burn-out, bore-out, migraine, Parkinson's disease, pulmonary hypertension, schizophrenia, an eating disorder, nausea, or vomiting.

The invention also refers to the use of a psilocin derivative as described herein in the manufacture of a medicament for the treatment of a serotonin 5-HT_{2A} receptor associated disease/disorder, preferably for the treatment of an anxiety disorder, attention deficit hyperactivity disorder (ADHD), depression, cluster headache, a condition associated with cancer, diminished drive, burn-out, bore-out, migraine, Parkinson's disease, pulmonary hypertension, schizophrenia, an eating disorder, nausea, or vomiting.

Moreover, the invention provides a method of treating a disease/disorder, particularly a serotonin 5-HT_{2A} receptor associated disease/disorder, in a subject in need thereof, the method comprising administering a therapeutically effective amount of the psilocin derivative according to the invention to said subject. It is preferred that the disease/disorder to be treated is an anxiety disorder, attention deficit hyperactivity disorder (ADHD), depression, cluster headache, a condition associated with cancer, diminished drive, burn-out, bore-out, migraine, Parkinson's disease, pulmonary hypertension, schizophrenia, an eating disorder, nausea, or vomiting.

In principle, the psilocin derivatives of formula (I) or the corresponding pharmaceutical compositions may be administered to a subject by any convenient route of administration. Various routes for administering pharmaceutical agents are known in the art and include, inter alia, oral (e.g., as a tablet, capsule, ovule, elixir, or as an ingestible solution or suspension), topical (e.g., transdermal, intranasal, ocular, buccal, and sublingual), parenteral (e.g., using injection techniques or infusion techniques, and including, for example, by injection, e.g., subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, or intrasternal by, e.g., implant of a depot, for example, subcutaneously or intramuscularly), pulmonary (e.g., by inhalation or insufflation therapy using, e.g., an aerosol, e.g., through mouth or nose), gastrointestinal, intrauterine, intraocular, subcutaneous, ophthalmic (including intravitreal or intracameral), rectal, or vaginal administration.

It is particularly preferred that the psilocin derivatives according to the invention (or corresponding pharmaceutical compositions) are administered orally, sublingually, or nasally (e.g., as a nasal spray or as nose drops). Suitable dosage forms for oral administration include, e.g., coated or uncoated tablets, soft gelatin capsules, hard gelatin capsules, lozenges, troches, solutions, emulsions, suspensions, syrups, elixirs, powders or granules for reconstitution, dispersible powders or granules, medicated gums, chewing tablets, or effervescent tablets. For oral administration, the psilocin derivatives or pharmaceutical compositions are preferably administered by oral ingestion, particularly by swallowing. The compounds or pharmaceutical compositions can thus be administered to pass through the mouth into the gastrointestinal tract, which can also be referred to as “oral-gastrointestinal” administration.

The subject or patient to be treated in accordance with the present invention may be an animal (e.g., a non-human animal). Preferably, the subject/patient is a mammal. More preferably, the subject/patient is a human (e.g., a male human or a female human) or a non-human mammal. Most preferably, the subject/patient to be treated in accordance with the invention is a human.

In this specification, a number of documents including patent applications/patents are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

The reference in this specification to any prior publication (or information derived therefrom) is not and should not be taken as an acknowledgment or admission or any form of suggestion that the corresponding prior publication (or the information derived therefrom) forms part of the common general knowledge in the technical field to which the present specification relates.

The invention will now be described by reference to the following examples which are merely illustrative and are not to be construed as a limitation of the scope of the present invention.

### EXAMPLES

### Example 1: Method of Production of psilocin-4-yl ethyl carbonate

Psilocin (2.1 mmol/429 mg) was suspended in tetrahydrofuran (100 ml) at 25° C. Triethylamine (5.0 mmol/0.7 ml) was added and aerated with argon. This results in a clear solution. Ethyl chloroformate (2.5 mmol/0.24 ml) was added dropwise through the septum. Upon addition, a whitish haze in the solution forms immediately. Stirring for 2.5 h under argon at 25° C.

In LC/MS at a wavelength of 225 nm, a sample of the reaction mixture showed almost complete reaction of the starting material. According to HPLC, 77% of product could be quantified.

The reaction is stirred for another hour at 25° C. under argon, followed by dilution with tetrahydrofuran (300 ml) to stop the reaction. The reaction mixture is concentrated on a rotary evaporator at 42° C. and subsequently dried at up to 10 mbar.

The raw product was taken up in 300 ml ethyl acetate and extracted with 200 ml 1 M hydrochloric acid, 200 ml water and 200 ml saturated saline solution. Subsequently, the organic phase was dried over some MgSO₄. Subsequently, the organic phase was slowly concentrated on the rotary evaporator, crystallizing the product from the solution as colorless crystals.

For purification, the material was recrystallized from isopropanol at 50° C. To strengthen the formation of crystals, some diisopropyl ether was added after cooling. Following filtration, 410 mg of colorless crystals were obtained.

The production of psilocin-4-yl ethyl carbonate can also be carried out as described above but using dichloromethane (instead of tetrahydrofuran) for suspending psilocin. Moreover, after the initial concentrating and drying steps, the extraction/washing step can also be skipped and, if desired, the raw product can instead be filtrated, e.g., through a small silica plug. For purification, the compound can also be stabilized as fumarate or oxalate salt and recrystallized in acetone.

### Example 2: Method of Production of psilocin-4-yl neopentyl carbonate

Psilocin (2.0 mmol/408 mg) was suspended in dichloromethane (12 ml) at 25° C. Triethylamine (2.6 mmol/0.36 ml) was added and aerated with argon. This results in a clear solution. Neopentyl chloroformate (2.1 mmol/0.32 ml) was added dropwise through septum. Upon addition, a whitish haze in the solution forms immediately. Stirring for 2.5 h under argon at 25° C.

The reaction was stopped by dilution with dichloromethane (40 ml). The desired crude product can be yielded by filtration through a small silica plug. For purification, the compound can be stabilized as fumarate or oxalate salt and recrystallized in acetone.

420 mg of oxalate salt were obtained as colorless crystals.

### Example 3: Method of Production of psilocin-4-yl benzyl carbonate

Psilocin (4.9 mmol/1.0 g) was suspended in dichloromethane (25 ml) at 25° C. Triethylamine (6.4 mmol/0.90 ml) was added and aerated with argon. This results in a clear solution. Benzyl chloroformate (5.4 mmol/0.80 ml) was added dropwise through septum. Upon addition, a whitish haze in the solution forms immediately. Stirring for 2.5 h under argon at 25° C.

The reaction was stopped by dilution with dichloromethane (60 ml). The desired crude product can be yielded by filtration through a small silica plug. For purification, the compound can be stabilized as fumarate or oxalate salt and recrystallized in acetone.

1.70 g of oxalate salt were obtained as colorless crystals.

### Example 4: Method of Production of psilocin-4-yl tryptophanate

Psilocin (1.5 mmol/300 mg) was suspended in tetrahydrofuran (100 ml) at 25° C. 4-Dimetyl amino pyridine (1.8 mmol/220 mg) and dicyclohexyl carbodiimide (1.8 mmol/370 mg) were added and aerated with argon. Stirring for 15 min at room temperature (RT). Fmoc-L-tryptophan (1.8 mmol/770 mg) was added. After addition, the suspension becomes clear and a solution is formed. Stirring for 5 h under argon at 25° C.

In LC/MS at a wavelength of 225 nm, a sample of the reaction mixture showed a reaction of 23%. According to HPLC, large amounts of psilocin and unreacted Fmoc-L-tryptophan could still be detected.

The reaction was stirred for another hour at 25° C. under argon, followed by dilution with tetrahydrofuran (200 ml) to stop the reaction. The reaction mixture is concentrated on the rotary evaporator at 42° C. and subsequently dried at up to 10 mbar.

The raw product was taken up in 200 ml ethyl acetate and extracted with 150 ml 1 M hydrochloric acid, 150 ml water and 150 ml saturated saline solution. Subsequently, the organic phase was dried over some MgSO₄. Subsequently, the organic phase was distilled off on the rotary evaporator, yielding the raw product as 1.2 g of yellow solid.

The raw product was treated on a column over 50 g silica using the eluent mixture hexane/ethyl acetate in a ratio of 7:3. This yielded 208 mg of this intermediate product as a colorless solid.

### Cleaving the Protective Group

The intermediate product was dissolved in 20 ml tetrahydrofuran at 25° C. Piperidine (0.7 mmol/59 mg) was added dropwise and aerated with argon. Stirring for 24 h at RT, and complete deprotection was shown by thin layer chromatography. The reaction mixture was concentrated on the rotary evaporator at 42° C. and then dried at up to 10 mbar. The raw product obtained was treated on a column over 20 g silica using the eluent mixture tert-butyl methyl ether/ethanol plus 1% ammonia in a ratio of 7:3. This yielded 106 mg as a nearly colorless solid.

### Example 5: Method of Production of psilocin-4-yl tert-butyl carbonate

The method of production of psilocin-4-yl tert-butyl carbonate is analogous to the method of production of psilocin-4-yl ethyl carbonate (see Example 1).

It comprises the steps:
a. preparing a suspension of psilocin in tetrahydrofuran;
b. adding triethylamine and 4-dimethylamino pyridine under protective gas atmosphere;
c. adding di-tert-butyl pyrocarbonate (dissolved in tetrahydrofuran) and
d. stirring the mixture under protective gas atmosphere for at least 3 hours;
e. stopping the reaction by diluting with tetrahydrofuran;
f. distilling off the tetrahydrofuran on the rotary evaporator and dissolving the residue in ethyl acetate;
g. extracting with 1 M HCl, water, and saturated saline solution;
h. drying the organic phase over a desiccant at 40-60° C. and under vacuum;
i. obtaining the raw product containing psilocin-4-yl tert-butylcarbonate;
j. recrystallizing from isopropanol.

### Example 6: Method of Production of psilocin-4-yl glycinate

The method of production of psilocin-4-yl glycinate is analogous to the method of production of psilocin-4-yl tryptophanate (see Example 4).

It comprises the steps:
a. preparing a suspension of psilocin in tetrahydrofuran;
b. adding 4-dimethylamino pyridine and 1-ethyl-3-(3-dimethylaminopropyl)carbodiimide hydrochloride;
c. adding N-carbobenzoxy glycine;
d. stirring the mixture under protective gas atmosphere for at least 5 hours;
e. stopping the reaction by diluting with tetrahydrofuran;
f. distilling off the tetrahydrofuran on the rotary evaporator and dissolving the residue in ethyl acetate;
g. extracting with 1 M HCl, water and saturated saline solution;
h. drying the organic phase over a desiccant at 40-60° C. and under vacuum;
i. obtaining the raw intermediate product containing psilocin-4-yl-Cbz glycinate;
j. purification by column chromatography using hexane/ethyl acetate;
k. cleaving the protective group by hydration using palladium on activated carbon in ethanol;
l. purification by column chromatography using tert-butyl methyl ether/ethanol 1% ammonia.

### Example 7: Method of Production of psilocin aspartate

The method of production of psilocin aspartate is analogous to the method of production of psilocin-4-yl tryptophanate (see Example 4).

It comprises the steps:
a. preparing a suspension of psilocin in tetrahydrofuran;
b. adding 4-dimethylamino pyridine and 1-ethyl-3-(3-dimethylaminopropyl)carbodiimide hydrochloride;
c. adding 4-benzyl N-carbobenzoxy L-aspartate;
d. stirring the mixture under protective gas atmosphere for at least 5 hours;
e. stopping the reaction by diluting with tetrahydrofuran;
f. distilling off the tetrahydrofuran on the rotary evaporator and dissolving the residue in ethyl acetate;
g. extracting with 1 M HCl, water, and saturated saline solution;
h. drying the organic phase over a desiccant at 40-60° C. and under vacuum;
i. obtaining the raw intermediate product containing psilocin-4-yl-N-Cbz benzyl aspartate;
j. purification by column chromatography using hexane/ethyl acetate;
k. cleaving the protective group by hydration using palladium on activated carbon in ethanol;
l. purification by column chromatography using tert-butyl methyl ether/ethanol 1% ammonia.

### Example 8: Solubility and Lipophilicity of psilocin/metocin carbonates

### Introduction

Three novel compounds (i.e., psilocin-4-yl ethylcarbonate, psilocin-4-yl tert-butylcarbonate, and N-methyl-N-ethyltryptamine-4-yl ethylcarbonate) were tested in vitro for their solubility in aqueous solution and for their lipophilicity, as compared to psilocybin and psilocin.

Aqueous solubility and lipophilicity can have important implications for pharmaceutical development. Firstly, both properties may affect the pharmacokinetics and bioavailability of the compounds in vivo. Secondly, these properties can help to determine the suitability of different compounds for development into different dosage forms.

### Methods

### Kinetic Turbidimetric Solubility Assay

Each test compound was diluted to 10 mM in DMSO. From this solution, six further dilutions of each test compound were prepared in DMSO (0.02, 0.1, 0.2, 1, 2, and 5 mM). Each of these solutions was then further diluted 1 in 50 in buffer (0.01 M phosphate buffered saline (pH 7.4)) so that the final DMSO concentration was 2% and the final test compound concentrations tested were 0.4, 2, 4, 20, 40, 100 and 200 μM. Due to the presence of visible particulates when psilocybin was diluted to 10 mM in DMSO, the seven final dilutions prepared for psilocybin instead were 0.2, 1, 2, 10, 20, 50 and 100 μM. A DMSO blank was also included. Three replicate wells were designated per concentration. Following the dilutions in buffer, plates were incubated at room temperature shaking for 5 minutes before the absorbance was measured at 620 nm using a Molecular Devices SpectraMax384 UV detector. Nicardipine was tested as a control compound. Psilocin-4-yl ethylcarbonate, psilocin-4-yl tert-butylcarbonate, and N-methyl-N-ethyltryptamine-4-yl ethylcarbonate were in salt form (hemifumarate), while psilocybin and psilocin were free base.

Solubility was estimated from the concentration of test compound that produced an increase in absorbance above a threshold of 0.005 absorbance units and was normalized to the DMSO blank.

### Micro Shake Flask Log D

10 mM solutions of each test compound were diluted in DMSO to give 400 μM solutions, which were then serially diluted into 2.5% DMSO in PBS to generate a calibration curve (0.014, 0.04, 0.12, 0.37, 1.11, 3.33 and 10 μM). 6 replicates of each test compound were incubated at 10 μM in a 1:9 ratio of Octanol: PBS at pH 7.4. Following a two hour incubation at room temperature shaking at 600 rpm, the incubation plate was centrifuged for 15 minutes to separate the layers and then two aliquots were removed from the PBS layer. The first was left neat and the second was diluted 10-fold to give dilute samples. Internal standards were added to both the calibration curve and the PBS incubation samples for analysis on LC MS/MS. Verapamil was tested as a control compound. Psilocin-4-yl ethylcarbonate, psilocin-4-yl tert-butylcarbonate, and N-methyl-N-ethyltryptamine-4-yl ethylcarbonate were in salt form (hemifumarate), while psilocybin and psilocin were free base.

Log D was measured as the concentration in the PBS layer against the generated calibration curve relative to the starting concentration of 10 μM. All 6 replicates of the neat samples were averaged to give one value for each, with the same calculation performed for the dilute sample values.

### Results

### Kinetic Turbiditimetric Solubility Assay

Particulates were visible when preparing the 10 mM stock solution of psilocybin in DMSO. All other compounds appeared to dissolve fully at 10 mM. For this reason, psilocybin was only tested at a maximum of concentration of 100 μM in the assay. By contrast, the solubility of the other compounds was able to be tested up to 200 μM.

TABLE 1
Maximum concentration of each compound tested in the solubility
assay. The compounds were soluble at the concentrations
shown. Note that psilocybin was tested at a lower maximum
concentration due to problems dissolving the compound
during preparation of the stock solution.

| | Maximum | | |
|---|---|---|---|
| Compound ID | concentration tested | Solubility | |
| Nicardipine | 200 μM | 24.4 | μM |
| Psilocybin | 100 μM | >100 | μM |
| Psilocin | 200 μM | >200 | μM |
| Psilocin-4-yl-ethylcarbonate | 200 μM | >200 | μM |
| Psilocin-4-yl-tert-butylcarbonate | 200 μM | >200 | μM |
| N-Methyl-N-Ethyltryptamine-4- | 200 μM | >200 | μM |
| yl-ethylcarbonate | | | |

### Micro Shake Flask Log D

TABLE 2
Mean LogD calculated for each compound using six
replicates in the Micro shake flask assay.

| Compound ID | Mean LogD |
|---|---|
| Verapimil | 2.49 |
| Psilocybin | 0.57 |
| Psilocin | 1.01 |
| Psilocin-4-yl-ethylcarbonate | 1.22 |
| Psilocin-4-yl-tert-butylcarbonate | 1.50 |
| N-Methyl-N-Ethyltryptamine-4-yl-ethylcarbonate | 1.01 |

### Conclusions

### Solubility

Psilocybin showed good solubility up to 100 μM, while the other novel compounds tested showed good solubility up to 200 μM. The challenge encountered while preparing the 10 mM stock solution of psilocybin supports the interpretation that the novel compounds tested exhibit greater aqueous solubility when compared to psilocybin.

### Lipophilicity

All novel compounds tested exhibited a Log D greater than psilocybin and greater than or equal to psilocin. The relatively low Log D of psilocybin may be consistent with limited permeability (Hartmann T and Schmitt J (2004) Lipophilicity—beyond octanol/water: a short comparison of modern technologies. Drug Discov Today 1(4): 431-439). By contrast, the log D>1 seen for the novel compounds is consistent with a range reported to be optimal for orally-administered CNS drugs (Kerns E H and Di L (2008) Drug-like properties: concepts, structure design and methods: from ADME to toxicity optimization. ISBN 0123695201 Academic Press). These results indicate that the psilocin carbonates according to the invention can pass the blood-brain barrier (BBB) faster than psilocybin, which makes them highly advantageous for therapeutic applications. Furthermore, the improved the solubility/lipophilicity profile of the novel compounds may enhance absorption of the prodrugs via passive diffusion when administered via non-oral routes, as compared with psilocybin.

### Example 9: Stability of Novel psilocin carbonates in HCl

### Introduction

Four novel compounds (i.e., psilocin-4-yl ethylcarbonate, psilocin-4-yl tert-butylcarbonate, psilocin-4-yl neopentylcarbonate, and psilocin-4-yl benzylcarbonate) were tested for their stability in 1% hydrochloric acid (HCl) as compared to psilocybin. These conditions were selected to provide insights into the chemical stability of compounds at a pH similar to gastric conditions.

### Methods

4 mg of each test compound was diluted in 2 ml distilled water to give a solution of 2 mg/ml. 2 ml of test compound solutions were then added to 2 ml of 2% (v/v) HCl in distilled water, yielding a final HCl concentration of 0.32 mM (pH 0.5). Test solutions were incubated at 37° C. with continuous stirring for approximately 26 hours. Concentrations of parent compound and psilocin were analyzed at various timepoints using LC-MS. Concentrations of both parent prodrug and psilocin liberated were expressed relative to the starting concentration of parent prodrug.

### Results

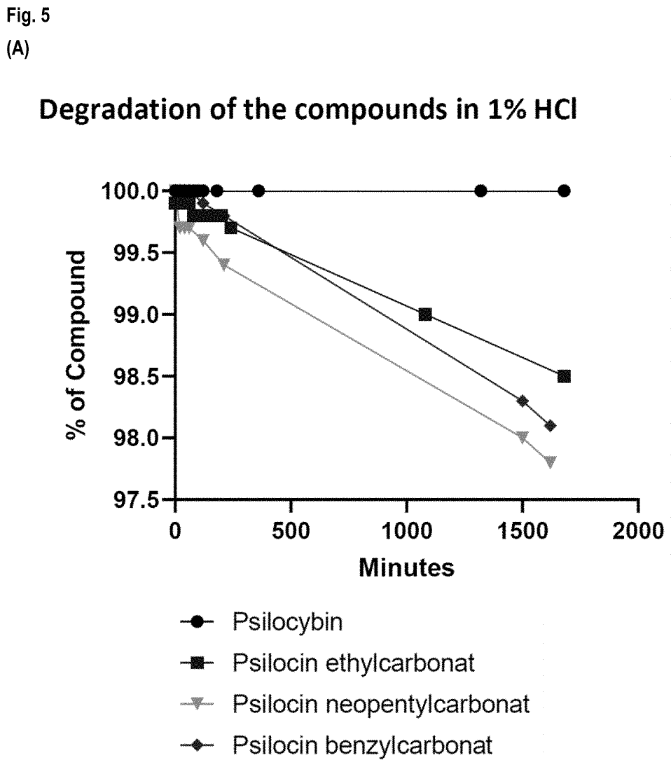

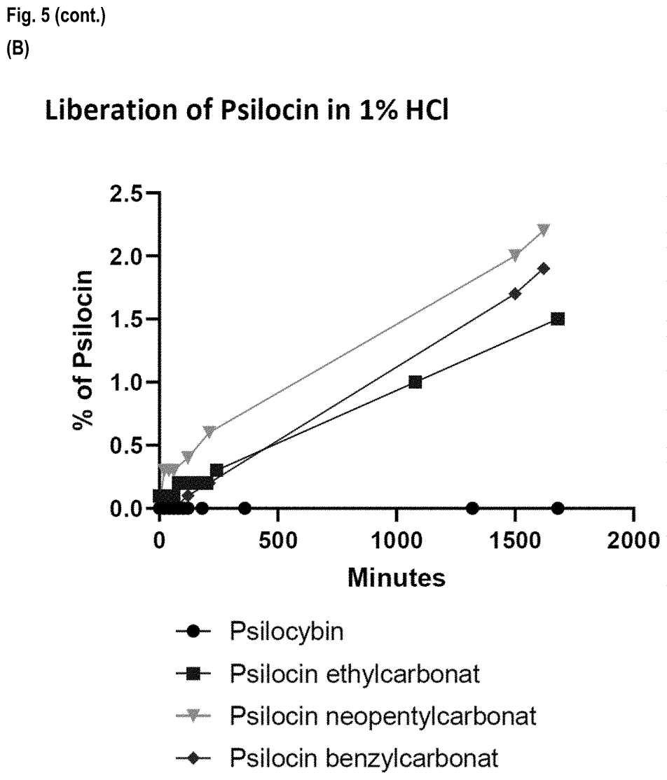

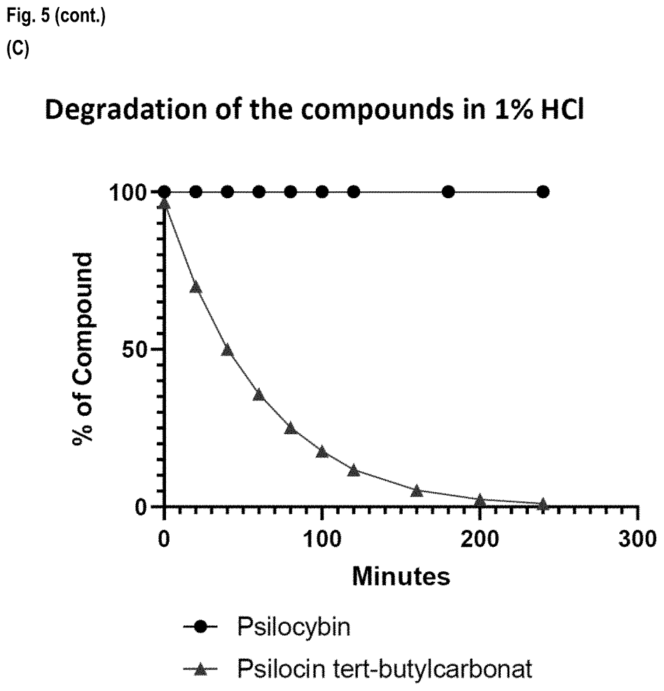

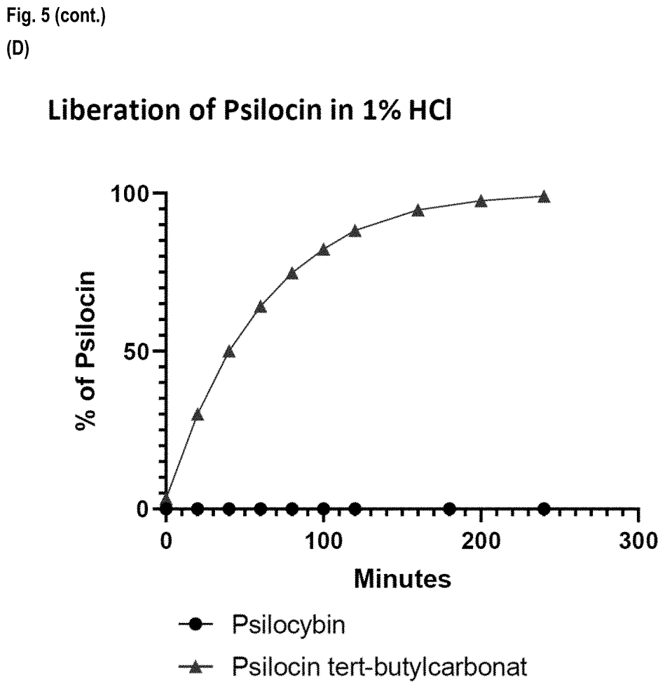

The results obtained in this experiment are shown in FIGS. 5A to 5D.

### Conclusions

In view of the above results, it has been found that all psilocin carbonates tested displayed greater degradation in 1% HCl than psilocybin, each successfully converting to psilocin under these conditions. Moreover, psilocin tert-butylcarbonate was especially susceptible to degradation by 1% HCl, showing rapid and complete chemical degradation to psilocin within approximately four hours of testing. These findings support the interpretation that psilocin carbonates generally show more rapid conversion to the active molecule psilocin under highly acidic conditions.

### Example 10: Intake Study

### Chemicals and Reagents

P1—Psilocin-4-yl ethylcarbonate (salt: hemifumarate)
P2—Psilocin-4-yl Cert-butylcarbonate (salt: hemifumarate)
M1—Prodrug of 5,6-methylenedioxy-2-aminoindane (MDAI) (salt: hemioxalate)
M2—Prodrug of 3,4-methylenedioxyamphetamine (MDA) (salt: hemioxalate)

Formic acid (Rotipuran® ≥98%, p.a.) and sodium fluoride (NaF, ≥99%, p.a) were obtained from Carl Roth (Karlsruhe, Germany). Acetonitrile (ACN, LC-MS grade), ammonium formate 10 M (99.995%), absolute ethanol, ascorbic acid (99%) and dimethyl sulfoxide (DMSO) were bought from Sigma Aldrich (Steinheim, Germany). Deionized water was prepared using a Medica® Pro deionizer from ELGA (Celle, Germany). Calf serum was obtained from Thermo Fisher Scientific (Waltham, USA). Mobile phase A (1% ACN, 0.1% HCOOH, 2 mM NH4⁺HCOO⁻ in water) and mobile phase B (0.1% HCOOH, 2 mM NH4⁺HCOO⁻ in ACN) were freshly prepared prior to analysis.

### Intake Study

Two volunteers (1 M, 1 F) ingested about 1 mg of each one of P1 and M1 (Study 1) and P2 and M2 (Study 2) in gelatin capsules (exact amounts in Tables 3 and 4). One blood sample was collected prior to ingestion of the substances and served as a zero control. After ingestion samples were collected for about 8 h with increasing time intervals between sample taking (exact protocol in Tables 4 and 5). Blood was collected in EDTA monovettes (Sarstedt, Nümbrecht, Germany) and centrifuged directly after collection (15 min, 4,000×g). The obtained sera were transferred in plastic tubes and NaF (ca. 30 mg) and ascorbic acid (ca. 5 mg) were added to increase analytes stability.

TABLE 3
Study design and sample collection protocol of Study 1

| | |
|---|---|
| A Female, 170 cm, 93 kg | B Male, 189 cm 113 kg |

| Appl. | | | Appl. | | |
|---|---|---|---|---|---|
| subst. | Salt | Free base | subst. | Salt | Free base |
| M1 | 1.05 mg | 935 μg | M1 | 0.99 mg | 881 μg |
| P1 | 1.03 mg | 852 μg | P1 | 0.99 mg | 819 μg |

| Sample | Time [min] | Sample | Time [min] |
|---|---|---|---|
| A1-0 | −33 | B1-0 | −25 |
| | 0 | | 0 |
| A1-1 | 7 | B1-1 | 6 |
| A1-2 | 15 | B1-2 | 13 |
| A1-3 | 21 | B1-3 | 20 |
| A1-4 | 31 | B1-4 | 30 |
| A1-5 | 49 | B1-5 | 47 |
| A1-6 | 65 | B1-6 | 64 |
| A1-7 | 87 | B1-7 | 83 |
| A1-8 | 120 | B1-8 | 114 |
| A1-9 | 155 | B1-9 | 165 |
| A1-10 | 165 | B1-10 | 232 |
| A1-11 | 240 | B1-11 | 322 |
| A1-12 | 338 | B1-12 | 369 |
| A1-13 | 371 | B1-13 | 432 |
| A1-14 | 424 | | |
| A1-15 | 486 | | |

TABLE 4
Study design and sample collection protocol of Study 2

| | |
|---|---|
| A Female, 170 cm, 93 kg | B Male, 189 cm 113 kg |

| Appl. | | | Appl. | | |
|---|---|---|---|---|---|
| subst. | Salt | Free base | subst. | Salt | Free base |
| M2 | 1.04 mg | 918 μg | M2 | 0.97 mg | 857 μg |
| P2 | 0.98 mg | 823 μg | P2 | 1.05 mg | 882 μg |

| Sample | Time [min] | Sample | Time [min] |
|---|---|---|---|
| A2-0 | −12 | B2-0 | −20 |
| | 0 | | 0 |
| A2-1 | 4 | B2-1 | 6 |
| A2-2 | 12 | B2-2 | 13 |
| A2-3 | 23 | B2-3 | 22 |
| A2-4 | 34 | B2-4 | 34 |
| A2-5 | 50 | B2-5 | 50 |
| A2-6 | 77 | B2-6 | 77 |
| A2-7 | 113 | B2-7 | 100 |
| A2-8 | 130 | B2-8 | 127 |
| A2-9 | 165 | B2-9 | 165 |
| A2-10 | 205 | B2-10 | 205 |
| A2-11 | 270 | B2-11 | 270 |
| A2-12 | 335 | B2-12 | 331 |
| A2-13 | 395 | B2-13 | 394 |
| A2-14 | 458 | B2-14 | 454 |

### Sample Preparation

200 μL serum were spiked with an internal standard solution (10 μL) and ACN (600 μL) was added to precipitate soluble proteins. Before mixing thoroughly ammonium formate (10 M, 200 μL) was added separate the aqueous from the organic phase. After centrifugation (6 min, 4,000×g), 500 μL of the organic phase were transferred into another vial and evaporated to dryness at 40° C. under a gentle stream of nitrogen. Samples were reconstituted in 100 μL mobile phase (A/B, 90/10, v/v) and used for analysis. For quantification a blank sample and a six point calibration (0.5, 1.0, 2.0, 5.0, 10, 20 ng/mL) in calf serum were prepared as described above.

### HPLC-MS/MS Analysis

The HPLC-MS system consisted of a Nexera X2 UHPLC system composed of three LC-30AD pumps, a DGU-30A3 degasser, a SIL-30AC autosampler, a CTO-10AS column oven and a CBM-20A controller (Shimadzu, Duisburg, Germany) coupled to a QTRAP 6500plus triple quadrupole linear ion trap mass spectrometer equipped with a TurbolonSpray Interface (Sciex, Darmstadt, Germany). The MS was operated in positive electrospray ionization mode. Data acquisition was performed in scheduled multiple reaction monitoring mode (detection window: 60 s) using Analyst software (version 1.7). MS parameters (declustering potential, entrance potential, collision energy, and collision cell exit potential) were optimized for all substances to obtain the best possible signal intensities (summary of the MRM parameters are given in Table 5). Ion source temperature and ion source voltage were set to 550° C. and +5500 V, respectively. Dwell time was 20 ms for every MRM transition. Curtain gas (N₂) pressure was 35 psi, ion source gas 1 and 2 (compressed air) pressure were 50 and 60 psi, respectively, and collision gas (N₂) pressure was set to “high.” Chromatographic separation was performed on a Kinetex® biphenyl column (100×2.1 mm, 2.6 μm particle size, Phenomenex, Aschaffenburg, Germany) with a corresponding guard column (SecurityGuard™ ULTRA Cartridges UHPLC Biphenyl for 2.1 mm ID columns, Phenomenex, Aschaffenburg, Germany). The autosampler and the column oven temperature were set to 10° C. and 40° C., respectively. The injection volume was 10 μL. Gradient elution with Mobile phase A and B was applied as shown in Table 6.

TABLE 5
HPLC-Gradient

| Time | % B |
|---|---|
| 0 | 10 |
| 3 | 30 |
| 4 | 50 |
| 6 | 75 |
| 6.5 | 95 |
| 7.5 | 95 |
| 8 | 10 |
| 10 | 10 |

TABLE 6
MRM-Parameters

| | | | | DP | EP | CE | CXP |
|---|---|---|---|---|---|---|---|
| Analyte | Rt [min] | Q1 [m/z] | Q3 [m/z] | [V] | [V] | [V] | [V] |
| LSD-D3 | 4.7 | 327.2 | 226.2 | 70 | 10 | 32 | 15 |
| MDA-D5 | 2.6 | 185.1 | 168.1 | 70 | 10 | 16 | 15 |
| MDMA-D5 | 2.9 | 199.1 | 165.1 | 70 | 10 | 13 | 15 |
| M1 1 | 5.1 | 364.2 | 347.2 | 70 | 10 | 19 | 15 |
| M1 2 | 5.1 | 364.2 | 176.2 | 70 | 10 | 26 | 15 |
| M1 3 | 5.1 | 364.2 | 159.2 | 70 | 10 | 32 | 15 |
| M2 1 | 5.2 | 366.2 | 130 | 70 | 10 | 22 | 15 |
| M2 2 | 5.2 | 366.2 | 159.2 | 70 | 10 | 34 | 15 |
| M2 3 | 5.2 | 366.2 | 220.3 | 70 | 10 | 23 | 15 |
| M3 1 | 5.3 | 396.2 | 193.3 | 70 | 10 | 32 | 15 |
| M3 2 | 5.3 | 396.2 | 379.3 | 70 | 10 | 19 | 15 |
| M3 3 | 5.3 | 396.2 | 208.2 | 70 | 10 | 30 | 15 |
| P1 1 | 4.2 | 277.2 | 58 | 70 | 10 | 50 | 15 |
| P1 2 | 4.2 | 277.2 | 160.2 | 70 | 10 | 32 | 15 |
| P1 3 | 4.2 | 277.2 | 232 | 70 | 10 | 22 | 15 |
| P2 1 | 5.1 | 305.2 | 205.2 | 70 | 10 | 25 | 15 |
| P2 2 | 5.1 | 305.2 | 249.2 | 70 | 10 | 16 | 15 |
| P2 3 | 5.1 | 305.2 | 160.2 | 70 | 10 | 50 | 15 |
| P3 1 | 4.6 | 291.2 | 72 | 70 | 10 | 22 | 15 |
| P3 2 | 4.6 | 291.2 | 232.3 | 70 | 10 | 24 | 15 |
| P3 3 | 4.6 | 291.2 | 160 | 70 | 10 | 37 | 15 |
| MDA 1 | 2.7 | 180.1 | 163.2 | 70 | 10 | 15 | 15 |
| MDA 2 | 2.7 | 180.1 | 135 | 70 | 10 | 26 | 15 |
| MDAI 1 | 2.3 | 178.1 | 161.1 | 70 | 10 | 17 | 15 |
| MDAI 2 | 2.3 | 178.1 | 131.1 | 70 | 10 | 26 | 15 |
| MDAI 3 | 2.3 | 178.1 | 103.1 | 70 | 10 | 37 | 15 |
| OH-DMT 1 | 2.2 | 205.1 | 160.1 | 70 | 10 | 21 | 15 |
| OH-DMT 2 | 2.2 | 205.1 | 115.1 | 70 | 10 | 45 | 15 |

Limits of detection (LOD) and limits of quantification (LOQ) were determined according to German DIN 32645. Concentrations of the applied calibration were 0.05, 0.10, 0.15, 0.20, 0.25, 0.30, 0.35, 0.40, and 0.45 ng/mL. LODs and LOQs are shown in Table 7.

TABLE 7
LOD and LOQ according to DIN 32645

| Analyte | LOD [ng/mL] | LOQ [ng/mL] |
|---|---|---|
| M1 | 0.12 | 1.1 |
| M2 | 0.08 | 0.46 |
| P1 | 0.05 | 0.30 |
| P2 | 0.07 | 0.27 |
| MDA | 0.13 | 0.50 |
| MDAI | 0.05 | 0.24 |
| Psilocin | 0.04 | 0.31 |

TABLE 8
Concentrations of analytes detected in
samples from test person A in study 1.

| Sample | Time [min] | c(MDAI) [ng/mL] | c(Psilocin) [ng/mL] |
|---|---|---|---|
| A1-0 | −33 | n.d. | n.d. |
| A1-1 | 7 | n.d. | n.d. |
| A1-2 | 15 | 0.36 | 0.15* |
| A1-3 | 21 | 0.606 | 0.34 |
| A1-4 | 31 | 0.936 | 0.51 |
| A1-5 | 49 | 1.74 | 0.82 |
| A1-6 | 65 | 1.61 | 1.45 |
| A1-7 | 87 | 2.82 | 1.02 |
| A1-8 | 120 | 2.64 | 1.02 |
| A1-9 | 155 | 2.54 | 0.59 |
| A1-10 | 165 | 2.77 | 0.98 |
| A1-11 | 240 | 2.02 | 0.58 |
| A1-12 | 338 | 1.79 | 0.34 |
| A1-13 | 371 | 1.66 | 0.33 |
| A1-14 | 424 | 1.4 | 0.29* |
| A1-15 | 486 | 0.974 | 0.26* |

TABLE 9
Concentrations of analytes detected in
samples from test person B in study 1.

| Sample | Time [min] | c(MDAI) [ng/mL] | c(Psilocin) [ng/mL] |
|---|---|---|---|
| B1-0 | −25 | n.d. | 0.22* |
| B1-1 | 6 | n.d. | 0.22* |
| B1-2 | 13 | n.d. | 0.25* |
| B1-3 | 20 | n.d. | 0.38 |
| B1-4 | 30 | n.d. | 0.60 |
| B1-5 | 47 | 0.61 | 0.76 |
| B1-6 | 64 | 1.24 | 0.54 |
| B1-7 | 83 | 0.932 | 0.60 |
| B1-8 | 114 | 1.21 | 0.43 |
| B1-9 | 165 | 1.98 | 0.37 |
| B1-10 | 232 | 1.35 | 0.32 |
| B1-11 | 322 | 0.83 | 0.25* |
| B1-12 | 369 | 0.763 | 0.24* |
| B1-13 | 432 | 0.609 | 0.22* |

*Concentrations were below the calculated LOQ and were extrapolated

TABLE 10
Cₘₐₓ, Tₘₐₓ, elimination half lives (t_{1/2}) and elimination constants
(kₑₗ) of psilocin and MDAI after oral intake of about 1 mg P1 and M1.

| | Psilocin (P1) | | MDAI (M1) | |
|---|---|---|---|---|
| | MEAN | SD | MEAN | SD |
| Cₘₐₓ (ng/mL) | 1.1 | 0.5 | 2.4 | 0.6 |
| Tₘₐₓ (min) | 56 | 12.73 | 126 | 55.15 |
| t_{1/2} (min) | 191 | 67 | 163 | 8.5 |
| kₑₗ (1/min) | 0.0039 | 0.0014 | 0.0043 | 0.00022 |

TABLE 11
Concentrations of analytes detected in
samples from test person A in study 2

| Sample | Time [min] | c(M2) [ng/mL] | c(Psilocin) [ng/mL] |
|---|---|---|---|
| A2-0 | −12 | n.d. | 0.17* |
| A2-1 | 4 | n.d. | 0.16* |
| A2-2 | 12 | n.d. | 0.16* |
| A2-3 | 23 | n.d. | 0.21* |
| A2-4 | 34 | n.d. | 0.22* |
| A2-5 | 50 | 0.02* | 0.43 |
| A2-6 | 77 | 0.05* | 0.57 |
| A2-7 | 113 | 0.14* | 0.99 |
| A2-8 | 130 | 0.17* | 1.14 |
| A2-9 | 165 | 0.14* | 0.96 |
| A2-10 | 205 | 0.15* | 0.83 |
| A2-11 | 270 | 0.13* | 0.66 |
| A2-12 | 335 | 0.06* | 0.47 |
| A2-13 | 395 | 0.05* | 0.40 |
| A2-14 | 458 | 0.02* | 0.30* |

*Concentrations were below the calculated LOQ and were extrapolated

TABLE 12
Concentrations of analytes detected in
samples from test person B in study 2.

| Sample | Time [min] | c(MDAI) [ng/mL] | c(Psilocin) [ng/mL] |
|---|---|---|---|
| B2-0 | −20 | n.d. | 0.25* |
| B2-1 | 6 | n.d. | 0.24* |
| B2-2 | 13 | n.d. | 0.26* |
| B2-3 | 22 | 0.03* | 0.48 |
| B2-4 | 34 | 0.1* | 0.75 |
| B2-5 | 50 | 0.29* | 1.12 |
| B2-6 | 77 | 0.32* | 1.12 |
| B2-7 | 100 | 0.22* | 0.92 |
| B2-8 | 127 | 0.14* | 0.73 |
| B2-9 | 165 | 0.06* | 0.53 |
| B2-10 | 205 | 0.04* | 0.51 |
| B2-11 | 270 | 0.02* | 0.40 |
| B2-12 | 331 | n.d. | 0.33 |
| B2-13 | 394 | n.d. | 0.30* |
| B2-14 | 454 | n.d. | 0.29* |

*Concentrations were below the calculated LOQ and were extrapolated

TABLE 13
Cₘₐₓ, Tₘₐₓ, elimination half lives (t_{1/2}) and elimination constants
(kₑₗ) of psilocin and M2 after oral intake of about 1 mg P2 and M2.

| | |
|---|---|
| Psilocin (P2) | M2 |

| | MEAN | SD | MEAN | SD |
|---|---|---|---|---|
| Cₘₐₓ (ng/mL) | 1.13 | 0.01 | 0.25 | 0.11 |
| Tₘₐₓ (min) | 90 | 57 | 104 | 38 |
| t_{1/2} (min) | 160 | 16 | 61 | 19 |
| kₑₗ (1/min) | 0.0043 | 0.00042 | 0.012 | 0.0038 |

### Conclusions

P1: After the oral ingestion of P1 only psilocin but no P1 itself (LOD 0.05 ng/mL) was detected in serum. The maximum concentration of psilocin (1.1±0.5 ng/mL) was observed 56±13 minutes after the application of P1. This is about one hour earlier as it is described after the oral application of psilocybin by Brown et al. (Brown R T et al., Pharmacokinetics of Escalating Doses of Oral Psilocybin in Healthy Adults, Clin Pharmacokinet (2017) 56:1543-1554, DOI:10.1007/s40262-017-0540-6). P1 is a fast releasing prodrug of psilocin in vivo.

P2: After the oral ingestion of P2 only psilocin but no P2 itself (LOD 0.07 ng/mL) was detected in serum. The maximum concentration of psilocin (1.13±0.01 ng/mL) was observed 90±56 minutes after application of P2. This is in the same range or bit earlier as it is described after the oral application of psilocybin by Brown et al. (loc. cit.). P2 acts a prodrug of psilocin in vivo with a potentially shorter Tₘₐₓ than psilocybin.

### Example 11: Solubility and Lipophilicity of Further psilocin carbonates

### Introduction

One novel compound (i.e., psilocin-4-yl-benzylcarbonate) was tested in vitro for solubility in aqueous solution and for lipophilicity.

Aqueous solubility and lipophilicity can have important implications for pharmaceutical development. Firstly, both properties may affect the pharmacokinetics and bioavailability of the compounds in vivo. Secondly, these properties can help to determine the suitability of different compounds for development into different dosage forms.

### Methods

### Kinetic Solubility Assay

Test and control compounds were diluted to 10 mM in DMSO and then further diluted 1 in 50 in 50 mM PB (pH 7.4) to a target concentration of 200 μM. Samples were vortexed for at least 2 minutes and then left to shake (800 rpm) at room temperature for 24 hours. Visual appearance was assessed before centrifugation and injection into a UPLC system to measure concentration. Carmbamezepine and Chloramaphenicol were run as control compounds. Each compound was tested in duplicate. Psilocin-4-yl-benzylcarbonate was tested as a hydrochloride salt.

### Shake Flask Log D Assay

Test and control compounds were diluted to 10 mM in DMSO. 2 μl of each stock solution was aliquoted into tubes in duplicate. 1-Octanol saturated phosphate buffer (PB) (pH 7.4) was prepared by adding 1-Octanol into 100 ml of 100 mM PB (7.4). PB saturated 1-Octanol was prepared by adding 10 ml of 100 mM PB (7.4) into 100 ml of 1-Octanol. 149 μl of each solution was aliquoted into the corresponding tubes. These were then mixed vigorously for 2 minutes and shaken (800 rpm) at room temperature for one hour. The appropriate volume of buffer and 1-Octanol layer samples was aliquoted and diluted, before detection using LC-MS/MS. Chlorpromazine, Nadolol, and Propranolol were run as control compounds. Psilocin-4-yl-benzylcarbonate, chlorpromazine and propranolol were tested as hydrochloride salts.

Log D was calculated according to the following equation:

$$Log {D}_{octanol / buffer} = log \frac{\begin{matrix}Mean Octanol Layer Peak Area Ratio * \\ Octanol Layer Dilution Factor\end{matrix}}{\begin{matrix}Mean Buffer Layer Peak Area Ratio * \\ Buffer Layer Dilution Factor\end{matrix}}$$

### Results

### Kinetic Solubility Assay

TABLE 14
Concentration of each compound tested in the solubility assay.
The compounds were soluble at the concentrations shown.

| | Target | Detected | |
|---|---|---|---|
| Compound ID | concentration | concentration | Solubility |
| Psilocin-4-yl- | 200 μM | 230 μM | >230 μM |
| benzylcarbonate | | | |
| Carbamazepine | 200 μM | 180 μM | >180 μM |
| Chloramphenicol | 200 μM | 197 μM | >197 μM |

### Shake Flask Log D

TABLE 15
Mean LogD calculated for each compound in the shake flask assay.

| Compound ID | Mean LogD |
|---|---|
| Psilocin-4-yl-benzylcarbonate | 2.19 |
| Chlorpromazine | 3.28 |
| Propranolol | 1.19 |
| Nadolol | −1.33 |

### Conclusions

### Solubility

Psilocin-4-yl-benzylcarbonate showed solubility up to >230 μM.

### Lipophilicity

Psilocin-4-yl-benzylcarbonate exhibited a Log D within the reported optimal range for orally-dosed CNS drugs (Kerns EH and Di L (2008) Drug-like properties: concepts, structure design and methods: from ADME to toxicity optimization, ISBN 0123695201, Academic Press). The Log D of psilocin-4-yl-benzylcarbonate was greater than that reported previously for psilocybin, indicating relatively greater lipophilicity and consequently a greater ability to pass the blood-brain barrier (BBB).

### Example 12: Pharmacokinetics of Novel psilocin carbonates in the Mouse

### Introduction

Three novel compounds (i.e., psilocin-4-yl ethylcarbonate, psilocin-4-yl tert-butylcarbonate, and psilocin-4-yl benzylcarbonate) were tested in mice to confirm their ability to release psilocin in vivo and to provide a comparison of their plasma psilocin pharmacokinetics to psilocybin.

### Methods

### Experimental Animals

27 male C57BL/6J mice weighing 22-25 g at time of purchase (Charles River UK) were group housed (3 s) in polypropylene cages. Mice were maintained on a normal phase 12 hr light-dark cycle (lights on from 07:00-19:00) with ad libitum access to standard pelleted diet (Envigo 2018) and filtered tap water. The holding room was maintained at 21±4° C. with a relative humidity of 55±15%.

### Experimental Procedures

Mice were weighed on the day of dosing and identified by a tail mark using a permanent marker pen. Food was not withdrawn on the day of dosing. Three animals were assigned to a control group that did not receive a treatment but were bled to enable collection of blank matrix. The remaining animals were divided into two cohorts, with animals in the first cohort receiving a single oral dose of one of the test compounds and animals in the second cohort receiving a single intravenous dose (in a lateral tail vein) of one of the test compounds. Following treatment, animals dosed orally were bled by venesection from the lateral tail vein at 5, 15, 30, 45, 60, 120, and 240 minutes post-dosing. Animals dosed intravenously were bled by venesection from the opposite lateral tail vein at the same timepoints. Psilocin-4-yl-ethylcarbonate, Psilocin-4-yl-tert-butylcarbonate, and Psilocin-4-yl benzylcarbonate were in salt form (hemifumarate), while psilocybin was free base. The dose of Psilocin-4-yl benzylcarbonate, but not Psilocin-4-yl ethylcarbonate or Psilocin-4-yl-tert-butylcarbonate, was corrected for salt weight. All compounds were formulated in saline and dosed in a volume of 5 ml/kg.

The dosing groups are summarised in the table below.

TABLE 16
Summary of dosing groups in the orally (PO)
and intravenously (IV) dosed cohorts.

| Test compound | Route | Dose | n |
|---|---|---|---|
| Psilocybin | PO | 0.3 mg/kg | 3 |
| Psilocin-4-yl-ethylcarbonate | PO | 0.3 mg/kg | 3 |
| Psilocin-4-yl-tert-butylcarbonate | PO | 0.3 mg/kg | 3 |
| Psilocin-4-yl benzylcarbonate | PO | 0.3 mg/kg | 3 |
| Psilocybin | IV | 0.3 mg/kg | 3 |
| Psilocin-4-yl-ethylcarbonate | IV | 0.3 mg/kg | 3 |
| Psilocin-4-yl-tert-butylcarbonate | IV | 0.3 mg/kg | 3 |
| Psilocin-4-yl benzylcarbonate | IV | 0.3 mg/kg | 3 |
| n/a (blank matrix) | — | — | 3 |

25 μl of blood was collected per animal per timepoint. Blood samples were collected into K2EDTA-coated tubes (e.g. Sarstedt Microvette 300K2E tubes) and kept on wet ice prior to centrifugation (10,000 RPM for 2 minutes). 10 μL of plasma was drawn off and placed in an individually identified Axygen mini tube. Samples were initially kept on dry ice and then transferred to a freezer at approximately −80° C. overnight. Blank matrix plasma samples were handled identically to test samples. Psilocin levels within samples were determined using LC-MS/MS. All pharmacokinetic parameters were calculated manually. Measurements below LLOQ (=2.5-5 ng/ml) were included as 0 ng/ml.

### Results

TABLE 17
Key parameters calculated for each test compound when
dosed via intravenous injection in the mouse. All
parameters correspond to psilocin measurement.

| | Dose | | Cₘₐₓ | Tₘₐₓ | |
|---|---|---|---|---|---|
| Compound | (mg/kg) | Route | (ng/ml) | (mins) | AUC₀₋ₜₗₐₛₜ |
| Psilocybin | 0.3 | IV | 81.1 | 5 | 37.5 |
| Psilocin-4-yl- | 0.3 | IV | 89.9 | 5 | 27.8 |
| ethylcarbonate | | | | | |
| Psilocin-4-yl-tert- | 0.3 | IV | 88.4 | 5* | 28.1 |
| butylcarbonate | | | | | |
| Psilocin-4-yl- | 0.3 | IV | 176 | 5 | 49.0 |
| benzylcarbonate | | | | | |

Notes:
IV, intravenous.
*Due to difficulty sampling, one animal was sampled at 7.5 mins instead of 5 mins. All animals receiving this compound exhibited Cₘₐₓ at the first successfully sampled timepoint.

TABLE 18
Key parameters calculated for each test compound
when dosed via oral gavage in the mouse. All parameters
correspond to psilocin measurement.

| | Dose | | Cₘₐₓ | Tₘₐₓ | |
|---|---|---|---|---|---|
| Compound | (mg/kg) | Route | (ng/ml) | (mins) | AUC₀₋ₜₗₐₛₜ |
| Psilocybin | 0.3 | PO | 21.7 | 30 | 22.0 |
| Psilocin-4-yl- | 0.3 | PO | 15.4 | 30 | 16.2 |
| ethylcarbonate | | | | | |
| Psilocin-4-yl-tert- | 0.3 | PO | 13.8 | 30 | 15.9 |
| butylcarbonate | | | | | |
| Psilocin-4-yl- | 0.3 | PO | 15.1 | 45 | 18.1 |
| benzylcarbonate | | | | | |

Notes:
PO, per os.

TABLE 19
% Absolute oral bioavailability (F) of psilocin
calculated for each test compound.

| | Dose | |
|---|---|---|
| Compound | (mg/kg) | % Bioavailability, F |
| Psilocybin | 0.3 | 59 |
| Psilocin-4-yl-ethylcarbonate | 0.3 | 58 |
| Psilocin-4-yl-tert-butylcarbonate | 0.3 | 56 |
| Psilocin-4-yl-benzylcarbonate | 0.3 | 37 |

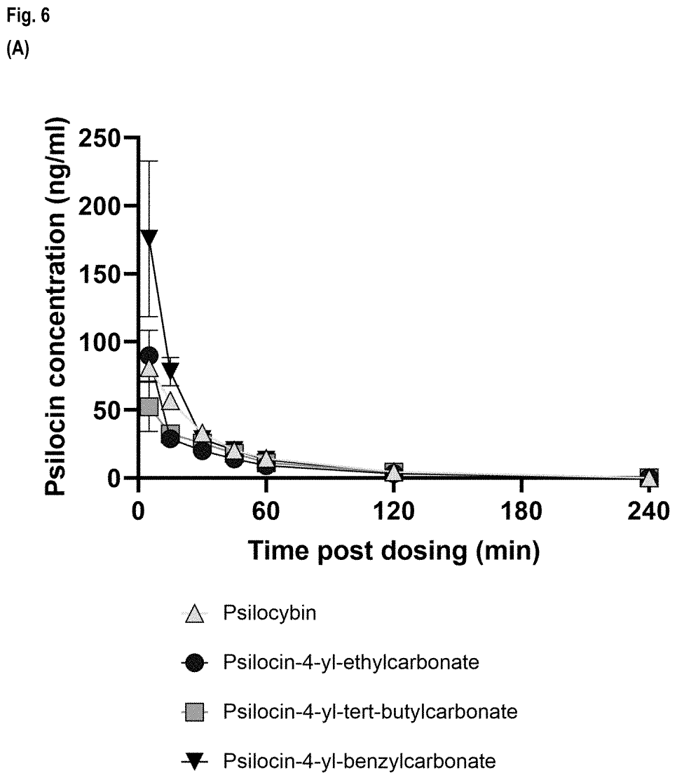

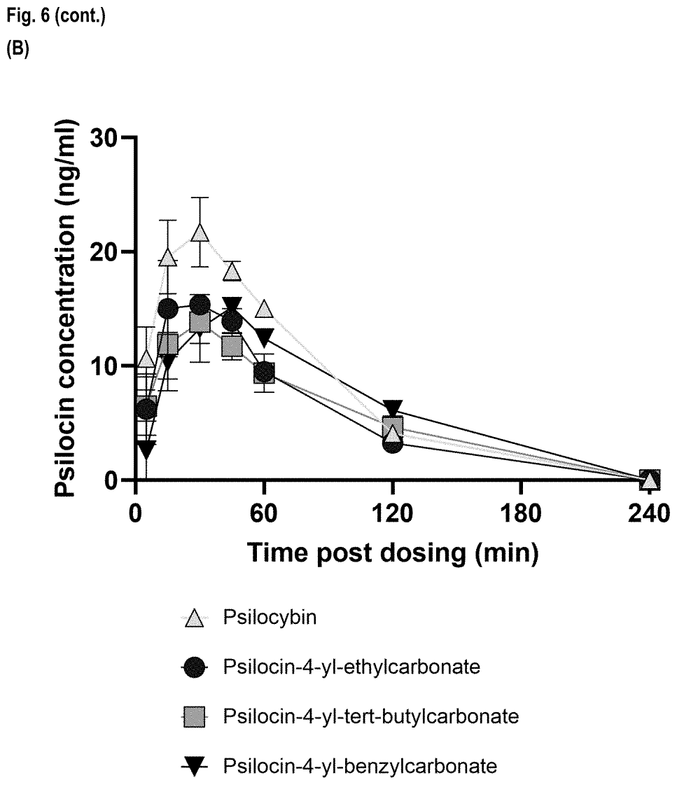

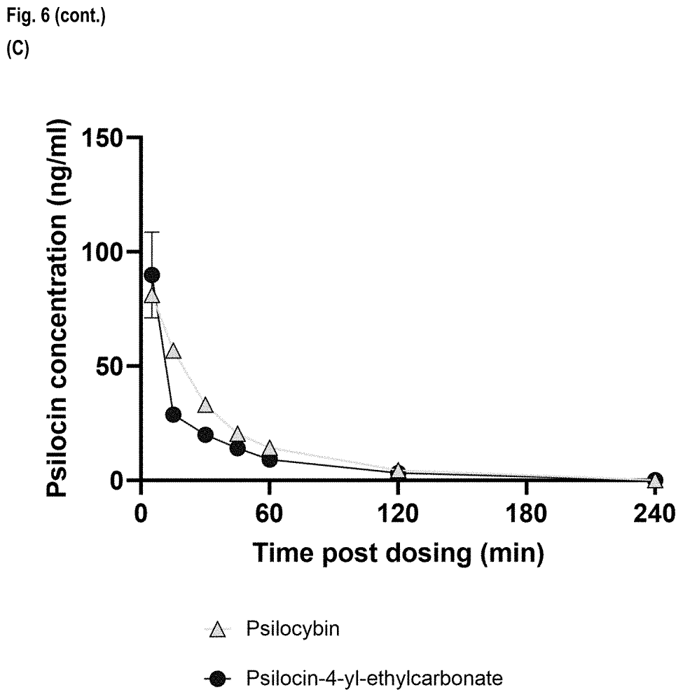

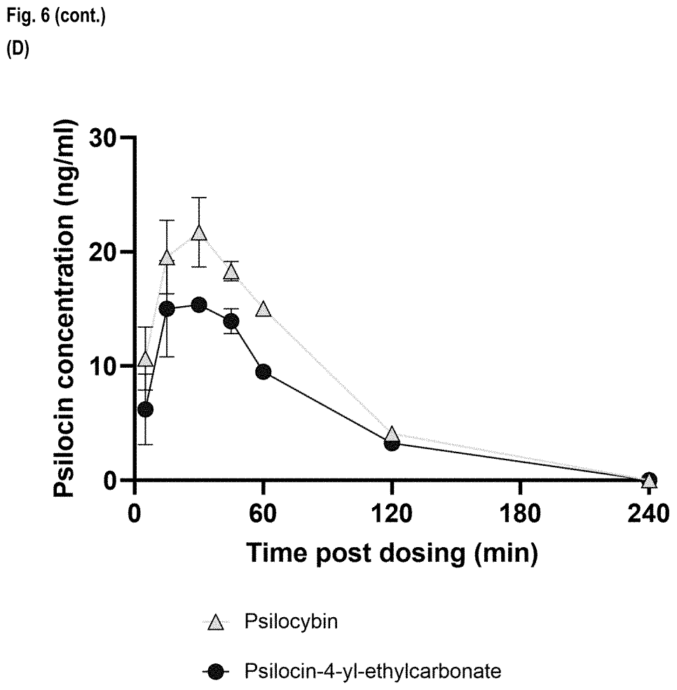

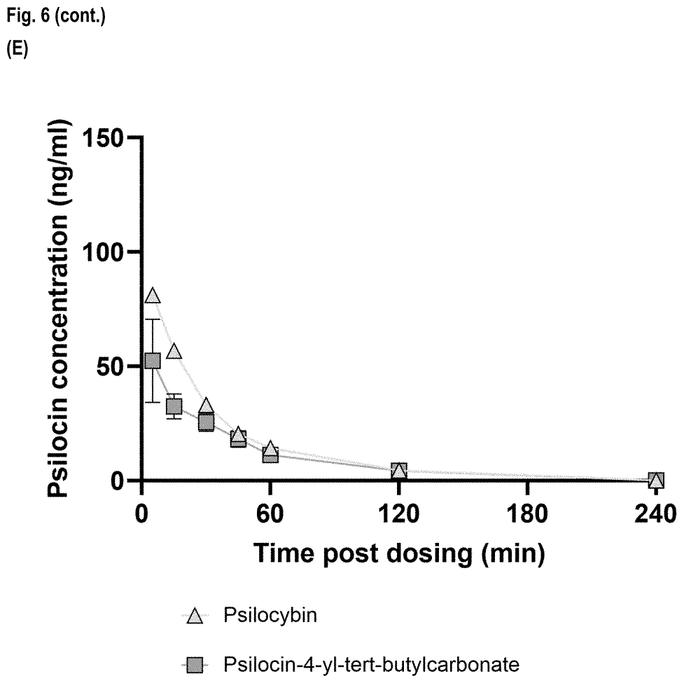

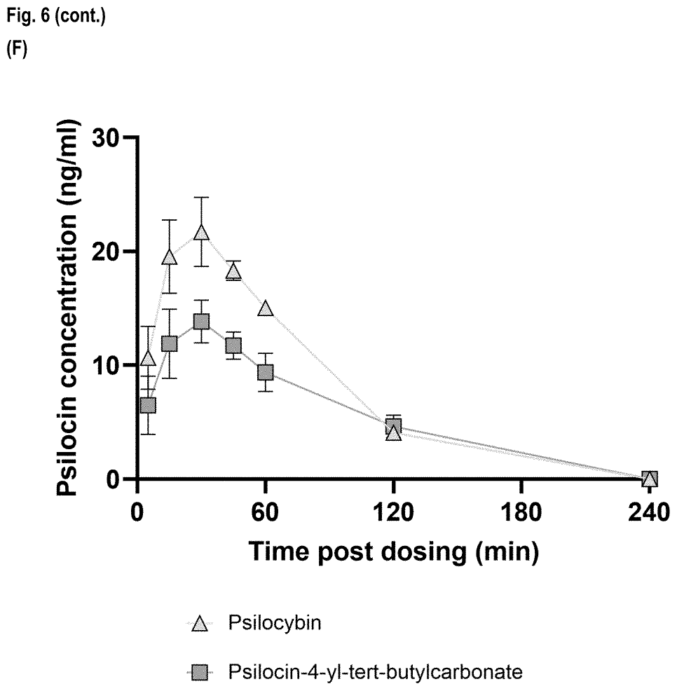

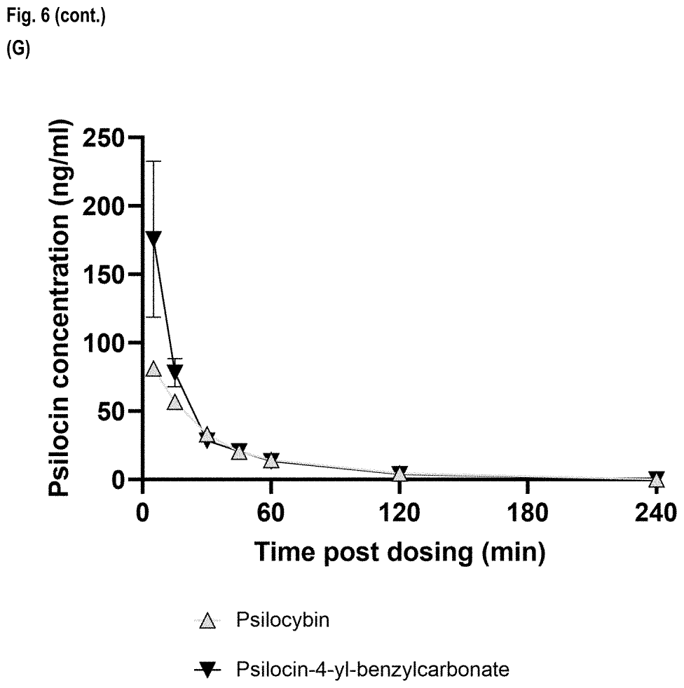

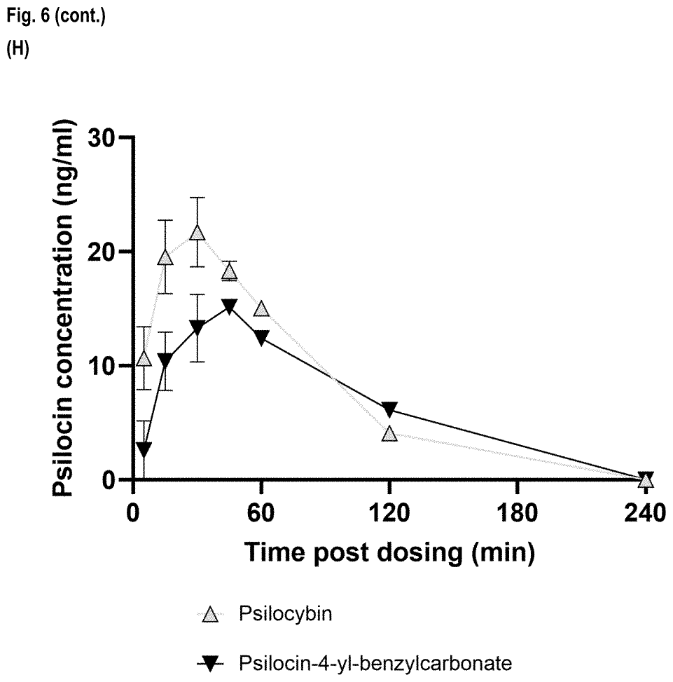

The plasma psilocin concentrations following intravenous dosing or oral dosing of mice with test compounds are shown in FIGS. 6A and 6B, respectively. Individual diagrams for each test compound are furthermore shown in FIGS. 6C to 6H, relating to plasma psilocin concentrations after psilocin-4-yl ethylcarbonate dosed intravenously (FIG. 6C) or orally (FIG. 6D), psilocin-4-yl tert-butylcarbonate dosed intravenously (FIG. 6E) or orally (FIG. 6F), and psilocin-4-yl benzylcarbonate dosed intravenously (FIG. 6G) or orally (FIG. 6H).

### Conclusions

### General Comments on IV Data

Psilocin was detected following dosing of all compounds, indicating conversion of each compound to psilocin in vivo when administered intravenously. When dosed intravenously, all novel compounds exhibited Cₘₐₓ similar or greater than psilocybin. In particular, psilocin-4-yl-benzylcarbonate showed a Cₘₐₓ approximately double that of psilocybin, resulting in a greater overall exposure as indicated by AUC. Tₘₐₓ was approximately equivalent between all compounds tested.

### General Comments on PO Data

Psilocin was detected following dosing of all compounds, indicating conversion of each compound to psilocin in vivo in the mouse when administered orally. When dosed orally, all novel compounds exhibited Cₘₐₓ slightly lower than psilocybin. Tₘₐₓ was approximately equivalent between all compounds tested (30 mins), with the exception of psilocin-4-yl-benzylcarbonate, which exhibited a delayed Tₘₐₓ (45 mins).

### Comments on psilocin-4-yl-benzylcarbonate IV vs Oral Administration

The relatively efficient conversion of psilocin-4-yl-benzylcarbonate to psilocin when dosed intravenously is in contrast to oral dosing of this compound (corresponding to relatively low oral % bioavailability), where Tₘₐₓ was slightly delayed compared to the other compounds tested and exposures were relatively low.

### Example 13: Head Twitch Response Induced by Novel psilocin/metocin carbonates

### Introduction

Four novel compounds (i.e., psilocin-4-yl ethylcarbonate, psilocin-4-yl tert-butylcarbonate, N-methyl-N-ethyltryptamine-4-yl ethylcarbonate, and psilocin-4-yl benzylcarbonate) and psilocybin were tested in mice for their ability to induce the head twitch response (HTR), an involuntary paroxysmal head rotation that occurs in rodents following activation of the serotonin 2A (5-HT_{2A}) receptor.

The HTR can be used to distinguish between psychedelic and non-psychedelic 5-HT_{2A} receptor agonists and, importantly, the potency of compounds for inducing the HTR in rodents is correlated to their potency for inducing psychedelic effects in humans (Halberstadt AL et al., Correlation between the potency of hallucinogens in the mouse head-twitch response assay and their behavioral and subjective effects in other species, Neuropharmacology (2020), doi: 10.1016/j.neuropharm.2019.107933).

### Methods

### Experimental Animals

48 male C57BL/6J mice weighing 20-25 g at time of purchase (Charles River UK) were group housed (3 s) in polypropylene cages. Mice were maintained on a normal phase 12 hr light-dark cycle (lights on from 07:00-19:00) with ad libitum access to standard pelleted diet (Envigo 2018) and filtered tap water. The holding room was maintained at 21±4° C. with a relative humidity of 55±15%.

### Experimental Procedures

Mice were weighed and allocated to a drug treatment group based on body weight. Animals were dosed via oral gavage with either vehicle, psilocybin (0.3 mg/kg), Psilocin-4-yl-ethylcarbonate hemifumarate (0.3 mg/kg), Psilocin-4-yl-tert-butylcarbonate hemifumarate (0.3 mg/kg), N-Methyl-N-Ethyltryptamine-4-yl-ethylcarbonate hemifumarate (0.3 mg/kg), or Psilocin-4-yl benzylcarbonate hemifumarate (0.3 mg/kg) and placed in a clean, clear cage containing a light layer of sawdust. The number of head twitches was then counted by a scorer blind to treatment condition for 60 minutes following dosing. Doses given for compounds in salt form were adjusted to ensure equivalent dose of drug (0.3 mg/kg) across all treatment groups. All compounds were formulated in saline and dosed in a volume of 5 ml/kg.

### Results

TABLE 20
Mean number of head twitches counted up to six
minutes post-dosing. N = 8 for all groups.

| | Mean number of head twitches | | | | |
|---|---|---|---|---|---|
| | | | | | Total |
| | 0-15 mins | 15-30 mins | 30-45 mins | 45-60 mins | 0-60 mins |
| Treatment group | (±SEM) | (±SEM) | (±SEM) | (±SEM) | (±SEM) |
| Vehicle | 3.5 | 1.75 | 1 | 0.38 | 5.54 |
| | (1.02) | (0.53) | (0.33) | (0.26) | (1.65) |
| Psilocybin | 4 | 3.75 | 3.13 | 1.25 | 10.83 |
| | (1.24) | (1.06) | (0.72) | (0.25) | (2.89) |
| Psilocin-4-yl- | 3.5 | 3.13 | 3.38 | 1.13 | 10.84 |
| ethylcarbonate | (0.53) | (0.52) | (0.75) | (0.23) | (1.13) |
| Psilocin-4-yl-tert- | 3 | 2 | 1.5 | 0.13 | 6.48 |
| butylcarbonate | (0.6) | (0.46) | (0.38) | (0.13) | (0.89) |
| N-Methyl-N- | 4.25 | 3.5 | 2.38 | 0.38 | 10.23 |
| Ethyltryptamine-4-yl- | (0.82) | (0.57) | (0.68) | (0.26) | (0.84) |
| ethylcarbonate | | | | | |
| Psilocin-4-yl | 2.63 | 4.13 | 1.88 | 1.13 | 8.2 |
| benzylcarbonate | (0.63) | (1.27) | (0.72) | (0.55) | (2.51) |

Notes:
SEM, Standard Error of the Mean.

### Conclusions

Over 60 mins post-dosing, psilocin-4-yl-ethylcarbonate and N-Methyl-N-Ethyltryptamine-4-yl-ethylcarbonate both induced a similar mean number of head twitches compared to psilocybin. Psilocin-4-yl-benylcarbonate also induced an Eₘₐₓ response during the 15-30 min time point of similar magnitude to that induced by psilocybin, as did N-Methyl-N-Ethyltryptamine-4-yl-ethylcarbonate at the 0-15 min time point. These findings support that 5-HT_{2A} receptor activation, which is required for psychedelic effects in humans, occurs following dosing of these compounds in the mouse.

1. A compound according to the general formula (I): 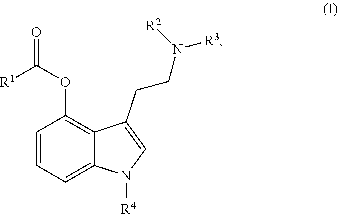
wherein:
R¹ is selected from —O—(C₁₋₁₂ alkyl), —O—CH₂-phenyl, —CH₂—NH₂, —CH(—NH₂)—CH₃, —CH(—NH₂)—CH(—CH₃)—CH₃, —CH(—NH₂)—CH₂—CH(—CH₃)—CH₃, —CH(—NH₂)—CH(—CH₃)—CH₂CH₃, —CH(—NH₂)—CH₂CH₂—S—CH₃, —CH(—NH₂)—CH₂—SH, —CH(—NH₂)—CH₂—OH, —CH(—NH₂)—CH(—CH₃)—OH, —CH(—NH₂)—CH₂—C(═O)—NH₂, —CH(—NH₂)—CH₂CH₂—C(═O)—NH₂, —CH(—NH₂)—CH₂—COOH, —CH(—NH₂)—CH₂CH₂—COOH, —CH(—NH₂)—CH₂CH₂CH₂CH₂—NH₂, —CH(—NH₂)—CH₂CH₂CH₂—NH—C(═NH)—NH₂, —CH(—NH₂)—CH₂-(1H-imidazol-4-yl), —CH(—NH₂)—CH₂-phenyl, —CH(—NH₂)—CH₂-(4-hydroxyphenyl), —CH(—NH₂)—CH₂-(1H-indol-3-yl), -(pyrrolidin-2-yl), -(4-hydroxypyrrolidin-2-yl), —CH(—NH₂)—CH₂—S—S—CH₂—CH(—NH₂)—COOH, —CH(—NH₂)—CH₂CH₂CH₂—NH₂, —CH(—NH₂)—CH₂CH₂CH₂—NH—C(═O)—NH₂, —CH₂—NH—CH₃, —CH(—NH₂)—CH₂CH₂—SH, —CH(—NH₂)—CH₂CH₂—OH, —CH(—NH₂)—CH₂-(3,4-dihydroxyphenyl), —CH(—NH₂)—CH₂-(5-hydroxy-1H-indol-3-yl), —CH₂CH₂—NH₂, —CH₂CH₂CH₂—NH₂, —CH(—CH₃)—CH₂—NH₂, —C(—NH₂)═CH₂, —O-(1-[R⁴]-3-[(-CH₂CH₂—N(—R²)—R³)]-1H-indol-4-yl), —O—(C₁₋₁₂ alkylene)-O-(1-[R⁴]-3-[(-CH₂CH₂—N(—R²)—R³)]-1H-indol-4-yl), —CH(—NH₂)—CH₂—COO-(1-[R⁴]-3-[(-CH₂CH₂—N(—R²)—R³)]-1H-indol-4-yl), —CH(—NH₂)—CH₂CH₂—COO-(1-[R⁴]-3-[(-CH₂CH₂—N(—R²)—R³)]-1H-indol-4-yl), —CH(—NH₂)—CH₂—S—S—CH₂—CH(—NH₂)—COO-(1-[R⁴]-3-[(-CH₂CH₂—N(—R²)—R³)]-1H-indol-4-yl), —O-(5-(aminomethyl)isoxazol-3-yl), and —CH(—NH₂)-(3-hydroxy-isoxazol-5-yl);
R² and R³ are each independently selected from hydrogen, methyl and ethyl, provided that R² and R³ are not both hydrogen; and
R⁴ is hydrogen or —C(═O)—O—(C₁₋₆ alkyl);
or a pharmaceutically acceptable salt thereof.

2. The compound according to claim 1, wherein R¹ is —O—(C₁₋₁₂ alkyl) or —O—CH₂-phenyl.

3. The compound according to claim 1, wherein R¹ is selected from —CH₂—NH₂, —CH(—NH₂)—CH₃, —CH(—NH₂)—CH(—CH₃)—CH₃, —CH(—NH₂)—CH₂—CH(—CH₃)—CH₃, —CH(—NH₂)—CH(—CH₃)—CH₂CH₃, —CH(—NH₂)—CH₂CH₂—S—CH₃, —CH(—NH₂)—CH₂—SH, —CH(—NH₂)—CH₂—OH, —CH(—NH₂)—CH(—CH₃)—OH, —CH(—NH₂)—CH₂—C(═O)—NH₂, —CH(—NH₂)—CH₂CH₂—C(═O)—NH₂, —CH(—NH₂)—CH₂—COOH, —CH(—NH₂)—CH₂CH₂—COOH, —CH(—NH₂)—CH₂CH₂CH₂CH₂—NH₂, —CH(—NH₂)—CH₂CH₂CH₂—NH—C(═NH)—NH₂, —CH(—NH₂)—CH₂-(1H-imidazol-4-yl), —CH(—NH₂)—CH₂-phenyl, —CH(—NH₂)—CH₂-(4-hydroxyphenyl), —CH(—NH₂)—CH₂-(1H-indol-3-yl), and -(pyrrolidin-2-yl).

4. The compound according to claim 1, wherein R¹ is selected from —CH(—NH₂)—CH(—CH₃)—CH₃, —CH(—NH₂)—CH₂—CH(—CH₃)—CH₃, —CH(—NH₂)—CH(—CH₃)—CH₂CH₃, —CH(—NH₂)—CH₂CH₂—S—CH₃, —CH(—NH₂)—CH₂—SH, —CH(—NH₂)—CH₂—OH, —CH(—NH₂)—CH(—CH₃)—OH, —CH(—NH₂)—CH₂—C(═O)—NH₂, —CH(—NH₂)—CH₂CH₂—C(═O)—NH₂, —CH(—NH₂)—CH₂—COOH, —CH(—NH₂)—CH₂CH₂—COOH, —CH(—NH₂)—CH₂CH₂CH₂CH₂—NH₂, —CH(—NH₂)—CH₂CH₂CH₂—NH—C(═NH)—NH₂, —CH(—NH₂)—CH₂-(1H-imidazol-4-yl), —CH(—NH₂)—CH₂-phenyl, —CH(—NH₂)—CH₂-(4-hydroxyphenyl), —CH(—NH₂)—CH₂-(1H-indol-3-yl), and -(pyrrolidin-2-yl).

5. The compound according to claim 1, wherein R¹ is selected from -(4-hydroxypyrrolidin-2-yl), —CH(—NH₂)—CH₂—S—S—CH₂—CH(—NH₂)—COOH, —CH(—NH₂)—CH₂CH₂CH₂—NH₂, —CH(—NH₂)—CH₂CH₂CH₂—NH—C(═O)—NH₂, —CH₂—NH—CH₃, —CH(—NH₂)—CH₂CH₂—SH, —CH(—NH₂)—CH₂CH₂—OH, —CH(—NH₂)—CH₂-(3,4-dihydroxyphenyl), —CH(—NH₂)—CH₂-(5-hydroxy-1H-indol-3-yl), —CH₂CH₂—NH₂, —CH₂CH₂CH₂—NH₂, —CH(—CH₃)—CH₂—NH₂, and —C(—NH₂)═CH₂.

6. The compound according to claim 1, wherein R² and R³ are each methyl.

7. The compound according to claim 1, wherein R² is methyl and R³ is hydrogen.

8. The compound according to claim 1, wherein R² is methyl and R³ is ethyl.

9. The compound according to claim 1, wherein R⁴ is hydrogen.

10. The compound according to claim 1, wherein R⁴ is —C(═O)—O—(C₂₋₄ alkyl).

11. The compound according to claim 1, wherein said psilocin derivative is a compound of the following formula: 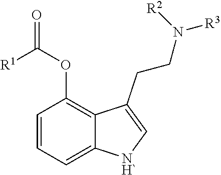
wherein:
R¹ is selected from —O—(C₂₋₅ alkyl), —O—CH₂-phenyl, —CH₂—NH₂, —CH(—NH₂)—CH₂—COOH, and —CH(—NH₂)—CH₂-(1H-indol-3-yl);
R² is methyl or ethyl; and
R³ is methyl or ethyl;
or a pharmaceutically acceptable salt thereof.

12. The compound according to claim 11, wherein R¹ is —O—(C₂₋₅ alkyl) or —O—CH₂-phenyl.

13. The compound according to claim 11, wherein R¹ is selected from —CH₂—NH₂, —CH(—NH₂)—CH₂—COOH, and —CH(—NH₂)—CH₂-(1H-indol-3-yl).

14. The compound according to claim 11, wherein R² and R³ are each methyl.

15. The compound according to claim 1, wherein said psilocin derivative is selected from any one of the following compounds or a pharmaceutically acceptable salt thereof:

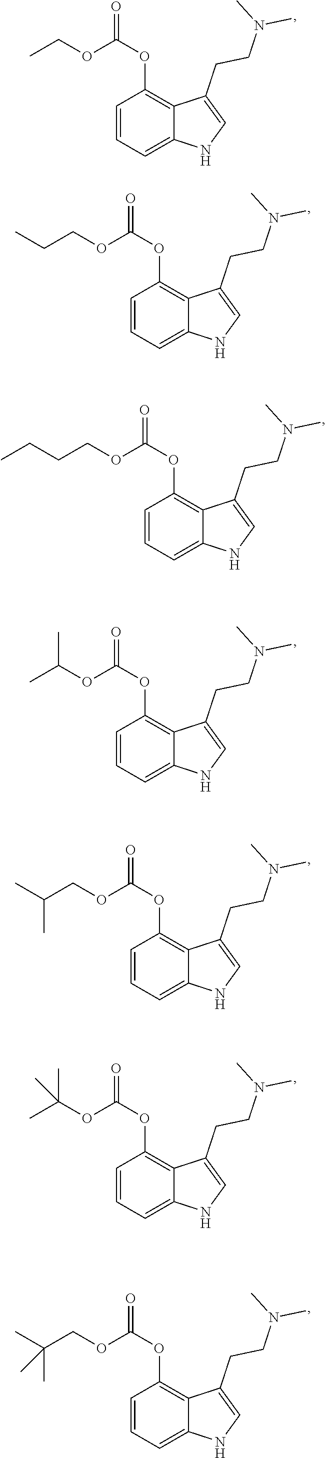

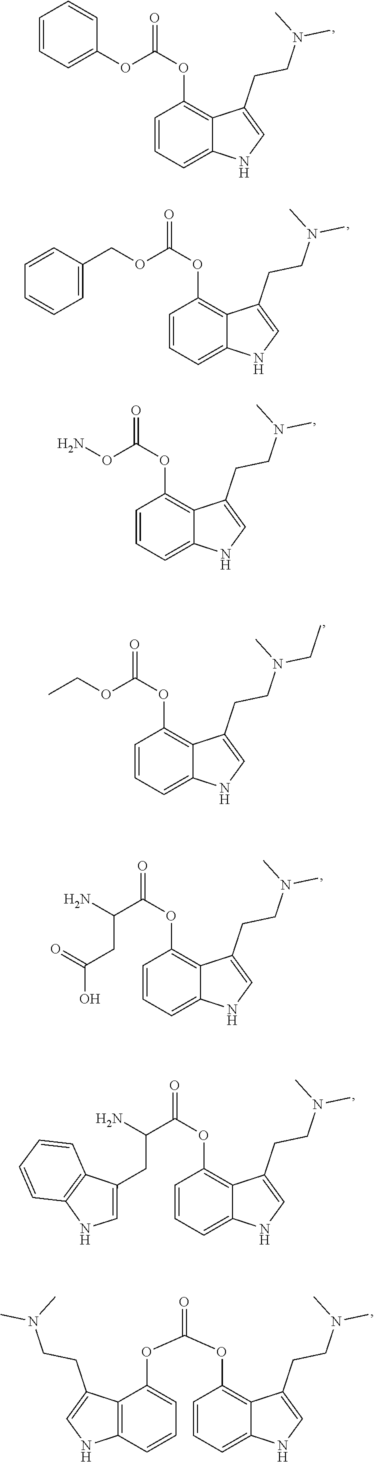

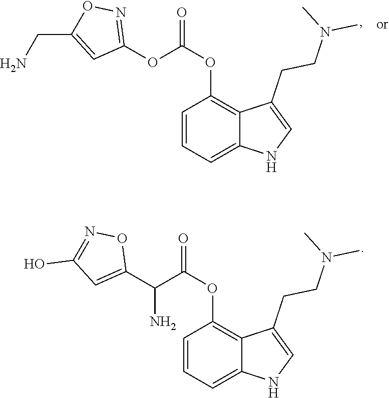 [SMILES: CN(C)CCc1c[nH]c2cccc(OC(=O)Oc3cc(CN)on3)c12 ; CN(C)CCc1c[nH]c2cccc(OC(=O)C(N)c3cc(O)no3)c12]

16. The compound according to claim 1, wherein said psilocin derivative is in the form of a pharmaceutically acceptable salt;
wherein said pharmaceutically acceptable salt is preferably a fumarate salt, a maleate salt, an oxalate salt, a malate salt, a tartrate salt, or a mesylate salt, more preferably an oxalate salt or a fumarate salt.

17. A pharmaceutical composition comprising at least one compound according to claim 1 and optionally one or more pharmaceutically acceptable excipients.

18.-21. (canceled)

22. A method of treating a serotonin 5-HT_{2A} receptor associated disease/disorder in a subject in need thereof, the method comprising administering a therapeutically effective amount of the compound according to claim 1 to said subject.

23. The method according to claim 22, wherein said disease/disorder is an anxiety disorder, attention deficit hyperactivity disorder (ADHD), depression, cluster headache, a condition associated with cancer, diminished drive, burn-out, bore-out, migraine, Parkinson's disease, pulmonary hypertension, schizophrenia, an eating disorder, nausea, or vomiting.

24. A method for producing a compound according to claim 1, comprising the steps of:
(a) preparing a suspension of psilocin in a solvent I;
(b) adding an activating agent under a protective gas atmosphere;
(c) adding a derivatization agent;
(d) stirring the mixture under a protective gas atmosphere for at least 3 hours;
(e) stopping the reaction by dilution with solvent;
(f) concentrating the solvent;
(g) dissolving the residue in a solvent II;
(h) extracting with 1 M HCl, water and saturated saline solution;
(i) drying the organic phase over a desiccant at 40-60° C. under vacuum;
(j) obtaining the crude product;
(k) purifying the crude product by recrystallization and/or column chromatography;
(l) obtaining the compound according to any one of claims 1 to 16.

25. The method according to claim 24, wherein:
(i) the activating agent is a nitrogen base, a carbodiimide, or a combination thereof; preferably wherein the activating agent is triethylamine, diisopropylethylamine, pyridine, 4-dimethylaminopyridine, 1-ethyl-3-(3′-dimethylaminopropyl)carbodiimide hydrochloride, dicyclohexylcarbodiimide, diisopropylcarbodiimide, or a combination thereof; and/or
(ii) the derivatization agent is ethyl chloroformate, di-tert-butyl pyrocarbonate, N-carbobenzoxy-glycine, N-(9-fluorenylmethyloxycarbonyl)-L-tryptophan, 4-benzyl N-carbobenzoxy-L-aspartate, N-carbobenzoxy-L-tryptophan, or N-benzyloxycarbonyl-L-tryptophan; and/or
(iii) the solvent I is tetrahydrofuran, 2-methyltetrahydrofuran, or dioxane; and/or
(iv) the solvent II is ethyl acetate, diethyl ether, dichloromethane, or a combination thereof; and/or
(v) the yield of the compound is at least 60 wt-% relative to the starting materials.
